(19) United States
(12) Patent Application Publication
(10) Pub. No.: US 2022/0344049 A1
(43) Pub. Date: Oct. 27, 2022

(54) DECENTRALIZED ARTIFICIAL INTELLIGENCE (AI)/MACHINE LEARNING TRAINING SYSTEM

(71) Applicant: PRESAGEN PTY LTD, Adelaide (AU)

(72) Inventors: Jonathan Michael MacGillivray HALL, Adelaide (AU); Donato PERUGINI, Adelaide (AU); Michelle PERUGINI, Adelaide (AU); Tuc Van NGUYEN, Adelaide (AU); Adrian JOHNSTON, Adelaide (AU)

(21) Appl. No.: 17/762,659

(22) PCT Filed: Sep. 23, 2020
(86) PCT No.: PCT/AU2020/000108

§ 371 Date: Mar. 22, 2022

(30) Foreign Application Priority Data
Sep. 23, 2019 (AU) 2019903539

(51) Int. Cl. G16H 50/20; G06N 3/04; G06N 3/08; G16H 30/40

(52) U.S. Cl. CPC G16H 50/20; G06N 3/0454; G06N 3/08; G16H 30/40

(57) ABSTRACT

A decentralized training platform is described for training an Artificial Intelligence (AI) model where training data (e.g., medical images) is distributed across multiple sites (nodes) and due to confidentiality, legal, or other reasons the data at each site is unable to be shared or leave the site and so cannot be copied to a central location for training. The method comprises training a teacher model locally at each node and then moving each of the teacher models to a central node and using these to train a student model using a transfer dataset. This may be facilitated by setting up the cloud service using inter-region peering connections between the nodes to make the nodes appear as a single cluster. In one variation the student module may be trained at each node using the multiple trained teacher models. In another variation we train multiple student models where each student model is trained by each teacher model at the node the teacher model was trained on, and once the plurality of student models are trained, an ensemble model is generated from the plurality of trained student models. Loss function weighting and node under sampling to enable load balancing may be used to improve accuracy and time/cost efficiency.

The present application is a U.S. national stage application of International Application Number PCT/AU2020/000108 filed on 23 Sep. 2020, which claims priority from Australian Provisional Patent Application No. 2019903539 titled “DECENTRALISED MACHINE LEARNING TRAINING SYSTEM” and filed on 23 Sep. 2019, the content of each application of which is hereby incorporated by reference in its entirety.

### BACKGROUND

### Technical Field

The present disclosure relates to Artificial Intelligence and machine learning computing systems. In a particular form the present disclosure relates to methods and systems for training AI/machine learning computing systems.

### Description of the Related Art

Traditional Computer vision techniques identify key features of an image and express this as a fixed length vector description. These features are often, “low-level” features such as object edges. These feature extract methods (SIFT, SURF, HOG, ORB, etc.) are hand-designed by researchers for each domain of interest (medical, scientific, general purpose images, etc.), with some level of overlap and re-usability. Typically, the feature extractors consist of a feature extraction matrix that is convolved over N×N patches of the image. The size of the patches is dependent on the technique used. However, it is impossible to hand craft accurate features that take into account more subtle cues such as texture and scene or background context.

Artificial Intelligence (AI), including Deep Learning and machine learning techniques, on the other hand, pose the problem of “learning” good features and representations—‘descriptions’ from large datasets. The current standard method in Computer Vision is to use a Convolutional Neural Network (CNN) to learn these feature representations. Similarly, to the feature extraction methods, a convolution is applied over the N×N image patches (size depends on the configuration). However, instead of hand crafting the weight matrix, the parameters of the convolution are optimized to achieve some goal, e.g., by computing a task dependent loss function (Classification, Segmentation, Object detection, etc.). Furthermore, instead of relying on a single convolution (or feature extraction), CNN's use multiple layered convolutions, where the extracted features of one layer are passed to the next convolution to be combined and extract the next feature representation. The exact network architecture (how the layers connect together) is dependent on the task and desired characteristics of the model (accuracy vs speed, training stability, etc.). This layered approach allows the model to learn to combine low-level features (like object edges similar to the simpler feature extraction methods) into more complicated representations which are often better for downstream tasks, such as Image Classification, Object Detection, Image Segmentation, etc., when compared with traditional methods. The general processing for training an Artificial Intelligence/machine learning model comprises cleaning and pre-processing of data (which may include labelling of outcomes in datasets), extraction of features (for example using computer vision libraries), selection of a model configuration (e.g., model architectures and machine learning hyper-parameters), splitting a dataset into training, validation and test datasets, training of the model using a deep learning and/or machine learning algorithms on the training dataset which involves modifying and optimizing model parameters or weights over a set of iterations known as epochs, and then selection or generation of the best model based on performance on the validation and/or test datasets.

A neural network is trained by optimizing the parameters or weights of the model to minimize a task-dependent ‘loss function.’ This loss function encodes the method of measuring the success of the neural network at optimizing the parameters for a given problem. For example, if we consider a Binary Image Classification problem, that is, separating a set of images into exactly two categories, the input images are first run through the model where a binary output label is computed, e.g., 0 or 1—to represent the two categories of interest. The predicted output is then compared against a ground truth label, and a loss (or error) is calculated. In the binary classification example, a Binary Cross-Entropy loss function is the most commonly used loss function. Using the loss value obtained from this function, we can compute the error gradients with respect to the input for each layer in the network. This process is known as back-propagation. The gradients are vectors that describe the direction in which the neural network parameters (or ‘weights’) are being altered during the optimization process in order to minimize the loss function. Intuitively, these gradients inform the network how to modify these weights to obtain a more accurate prediction for each of the images. In practice however, it is impossible to compute the network update in a single iteration or ‘epoch’ of training. Often, this is due to the networks requiring a large amount of data and containing a large number of parameters that can be modified. To solve this, often, mini-batches of data are used in place of the full set. Each of these batches is drawn at random from the dataset, and a large enough batch size is chosen to approximate the statistics for the entire dataset. The optimization then is applied over the mini-batches until a stopping condition is met (i.e., until convergence, or satisfactory results according to a pre-defined metric are achieved). This process is known as Stochastic Gradient Decent (SGD) and is the standard process of optimizing neural networks. Usually, the optimizer is run for hundreds of thousands to millions of iterations. Furthermore, neural network optimization is a ‘non-convex’ problem, which means that there are often many local minima in the parameter space defined by the loss function. Intuitively, this means that due to the complex interactions among the weights in the network and the data, there are many almost-equally valid combinations of weights that result in almost-identical outputs.

Deep Learning models, or neural network architectures that contain many layers of CNNs, are typically trained using Graphics Processing Units (GPUs). GPUs are extremely efficient at computing Linear Algebra compared with Central Processing Units (CPUs). As a result, they have found heavy use in High Performance Computing (HPC), and especially in training Neural Networks.

One limitation of Deep Learning methods is that they require large amounts of data to train from initialization (100K+ samples). This is due to the large number of parameters that the models contain, which is in the order of 1 million to 1 billion parameters, depending on the model and task. These parameters are then tuned, or optimized, from a random initialization. The best randomization strategy to use is often task specific and network specific, and there are many best practices that can be followed for setting the initialization values. However, when insufficient data are accessible, ab initio training often leads to ‘overfitting’ the data. This means that the model performs well on the data that is trained on, but fails to generalize to new unseen data. This is usually due to over-parameterization, i.e., there are too many parameters in the model suitable for the fitting problem and therefore it has memorized or overfit to the training examples. Techniques used to combat overfitting are typically called ‘regularization’ techniques.

When there is insufficient data (e.g., less than 100K examples), which is often the case for medical images or other applications where high data integrity is a premium resource, rather than start from a random initialization, it is possible to start from a model that has already been trained on another larger dataset. This method is called ‘pretraining.’ This has a regularization effect, and allows the models to be trained on a smaller amount of data, whilst maintaining optimal performance (sufficiently minimized loss function). Furthermore, the features learned from this dataset are general, and often translate well to data from new domains. For example, models can be pretrained on ImageNet (a general, publicly available image dataset) and then fine tune the model on a medical dataset. This process is known as either ‘fine-tuning’ or ‘transfer learning,’ and these terms are often used interchangeably.

A simple way to improve the performance of a neural network is to increase the number of layers in the model. Many of the most recent state of the art models contain more parameters than can reasonably fit into a single GPU. Furthermore, as training deep learning models requires a large amount of data and a large amount of iterative updates, it became necessary to utilize multiple GPUs and multiple machines. This process is known as Distributed Training.

When performing distributed training, a distribution strategy needs to be chosen. This defines how the workload will be divided among the different worker nodes. The two methods for this are Model Parallelism and Data Parallelism. Model Parallelism splits the workload by segmenting the model weights into N partitions, where N is the number of workers amongst which to split the work. Each segment then processes sequentially one after the other, with intermediate values being communicated via some form of network connection. This method can be useful when there is an obvious asynchronicity between sections of a model. For example, consider a model that takes two inputs of different modalities (images and audio). Each of these inputs can processed independently, before being joined together at a later stage of the model. However, this process is not necessarily more efficient standard training as network transmission costs can outweigh the improvement in computational performance. Therefore, Model Parallelism is typically better to scale the size of the model, e.g., by using models that contain more parameters than would fit on a single machine/GPU. Data Parallelism on the other hand, instead splits the data into N partitions. More specifically, the minibatch is split into N even groups. A copy of the current model is placed on each of the worker nodes, i.e., the dataset itself is replicated on each computer prior to training, the forward passes of the models are then performed parallel and the loss for each of the batches is then computed. The backward pass of the model is then computed afterwards. This consists of sequentially computing the gradients for each of the layers of the model. There are several methods for this with the most common method being reverse mode (reverse accumulation) Automatic Differentiation.

In both Model Parallel and Data Parallel frameworks, the gradients for each of the nodes need to be synchronized such that the weights can be updated in the final step of SGD. There are two main methods for doing this, which are: Parameter Server and Ring All-Reduce.

In the Parameter Server case, one of the worker nodes is selected to be the ‘master.’ The master node functions as a normal worker node with the additional role of combining together the results of the other nodes, to form a single model, and then update each of the worker nodes. Each of the gradients for each of the workers is then computed locally to that node, and then each of the N workers transmits the gradients for their sub-batch to the master node. The master node then averages the gradients for each of the nodes to obtain the final gradient update. Finally, the master node updates its weights via the chosen gradient decent algorithm (e.g., SGD) and then transmits the new weights to each of the worker nodes, so that at the end of each batch, each node has a copy of the full model incorporating all weight updates from every other node.

Ring All-Reduce on the other hand, does not have a master node. After the forward pass, each node computes its loss and gradients as usual. However, instead of communicating the gradients only to the master, each worker sends the gradients to adjacent workers in a peer-to-peer fashion. Every worker independently then averages the gradients it has received in parallel and updates using the chosen gradient decent algorithm. Compared with a Parameters Server, this process results in superior training scalability in terms of absolute numbers of workers. However, Ring All-Reduce requires a significant increase in overall network traffic as each node must transmit gradients to every other node in the work-group.

The characteristics of Distributed Training versus standard training on a single node are such that the performance is roughly equivalent in terms of the final trained model accuracy, but the total training time scales linearly with the number of nodes in the cluster. For example, using 100 Nodes will speed up the training process by 100 times.

In the machine learning literature, Federated Learning is a process of training a model using decentralized data and decentralized computation, e.g., multiple processors inside phones that contribute to a machine learning model. This is mainly done for privacy reasons, which is necessary for the use of processors and devices which are not owned or administered by the agent carrying out the training of the model. However Federated learning is typically done at the individual data point level and as such it requires the use of heavy encryption protocols because AI weights that are shared up to the central point can be used to infer personal information as they are based on an individual data point (or person), which considerably slows and complicates the learning process. Hence Federated Learning is usually used in the context of deploying and updating (or iterating over) an already trained AI. Federated Learning can be used to allow remote workers to contribute to the training phase of a machine learning model, without disclosing their datasets to the Master Node, and protects the model weights for each remote child model, but as noted such a learning process is very slow. The term Federated Learning is also sometimes used interchangeably with the term Decentralized Training. However in this document, the term Decentralized Training will be used in the case where training of the model is performed on distributed data where data privacy is required to the extent such that:

(1) data is not moved or copied from its own locality, and must remain in its locality during training; and

(2) the trained AI model that is shared does not contain a copy or substantive copy of the data being trained on but only contains a general derivative of the data.

Another approach in AI and machine learning is known as ‘Knowledge Distillation’ (shortened to Distillation) or ‘Student-Teacher’ models in which the distributions of the weight parameters obtained from one (or multiple) models (Teacher(s)) are used to inform the weight updates of another model (Student) via the loss function of the Student model. We will use the term Distillation to describe the process of training a Student model using Teacher model(s). The idea behind this procedure is to train the Student model to mimic a set of Teacher model(s). The intuition behind this process, is that the Teacher models contain subtle but important relationships between the predicted output probabilities (soft labels) that are not present in the original predicted probabilities (hard labels) obtained directly from the model results in the absence of the distributions from the Teacher model(s).

First, the set of Teacher model(s) are trained on the dataset of interest. The Teacher models can be of any neural network or model architecture, and can even be completely different architectures from each other or the Student model. They can either share the same dataset exactly, or have disjoint or overlapping subsets of the original dataset. Once the Teacher models are trained, the Student is trained using a distillation loss function to mimic the outputs of the Teacher models. The distillation process begins by first applying the Teacher model to a dataset that is made available to both the Teacher and Student models, known as the ‘transfer dataset.’ The transfer dataset can be hold-out, blind dataset drawn from the original dataset, or could be the original dataset itself Furthermore, the transfer dataset does not have to be completely labelled, i.e., with some portion of the data not associated with a known outcome. This removal of the labelling restriction allows for the dataset to be artificially increased in size. Then the Student model is applied to the transfer dataset. The output probabilities (soft labels) of the Teacher model are compared with the output probabilities of the Student model via a divergence measure function, such as Kullback-Leibler (KL)-Divergence, or ‘relative entropy’ function, computed from the distributions. A divergence measure is an accepted mathematical method for measuring the “distance” between two probability distributions. The divergence measure is then summed together with a standard cross-entropy classification loss function, so that the loss function is effectively minimizing both the classification loss, improving model performance, and also the divergence of the Student model from the Teacher model, simultaneously. Typically, the soft label matching loss (the divergence component of the new loss) and the hard label classification loss (the original component of the loss) are weighted with respect to each other (introducing an extra tuneable parameter to the training process) to control the contribution of each of the two terms in the new loss function.

Artificial Intelligence (AI), including deep learning and machine learning computing systems involve learning or training of the AI on large datasets. In particular, it is commercially and/or operationally important to build/train AI that is both accurate and robust, i.e., AI that is general, transferable and without bias such that it can accurately be applied to the intended (specific) problem. For applications in data analysis from the health industry, this includes (but not limited to) any clinical setting, demographic, country, hardware setup, etc. This is the case within and beyond health-related applications. To build AI that is accurate and robust, the AI needs to be trained on a dataset that is both large and diverse, typically from many data-sources distributed globally, e.g., in the case of health from clinics or hospitals distributed all over the world.

However due to data privacy, confidentiality, security, regulatory/legal, or technical reasons, it is not always possible to collect or transfer the data into a single location to create one large and diverse global dataset for the AI to train on. For example health regulation may not allow private medical data to legally leave the country of origin. This prevents training AI on a global dataset, and thus impact on the accuracy and robustness of the resulting AI that can only be trained on local datasets. This also impacts on the commercial viability of the AI that is produced, in particular its scalability. When the AI trained on a smaller and local dataset is produced and is scaled globally then it will either: (1) slow the commercialization efforts since the AI will need to be retrained in each location, region or dataset; or (2) the AI may break/fail in operational use by encountering a new situation or context that was not seen before in its training.

For example developing AI systems for health and medical applications is often difficult due to a lack of data sharing between hospitals, clinics and other medical institutes. This makes sense for the following reasons: the need for patient data to remain private (Data Privacy), the need for business records, corporate IP and business value assets to remain confidential within a clinic (Confidentiality) and the demands of legislation and regulation on records of a sensitive nature (Regulation). These issues are not only true in the field of medicine, but also extend to other industries, such as defense and security, or other businesses which contain or rely on confidential information. While the confidential information itself cannot be shared among institutes, there is a large amount of inherent value in the combined learnings of all the data across multiple sources, which would greatly benefit each industry by being able to apply new, well-tested (and testable) machine learning models that are robust. The creation and testing of a robust AI model can only occur if it is possible to develop methodologies that are able to leverage the power of the combined data sets, without removing or forcing a disclosure of the data themselves.

The above problem comes into play most frequently in the topic of data locality, where relevant data useful for constructing novel machine learning tools for an industry are distributed across multiple institutes, and no single source contains sufficient data for training a model that can generalize well to unseen data sets, or in some cases for training any reliable model at all, even for its own locality.

There are a number of methods for overcoming this barrier, where the industry need for a machine learning model outweighs the risk of encountering Data Privacy, Confidentiality or unsurpassable Regulatory roadblocks. First, it may be agreed among institutions that a certain portion of their data will be deemed suitable for making available to each other or a third party, which can facilitate the building of a shared model. However, this is not a process that will be suitable for sensitive data in general, and still places stringent limits on the type of data and amount of data available for the training of the models. This is also a time-consuming process, in which institutes must be convinced individually to release data.

Distributed Training systems thus provide a method for overcoming the problem of distributed data. For example, in training a machine learning model, institutes could choose not to release the data at all, but to make it available on a secure server or computer assigned to them, which no other institute can access, such as a cloud-service or local machine or portable device. On this server, a training process can run in a distributed manner, and multiple institutes can run concurrently, with only the updates to the machine learning model being shared, using the methodology described above in in Computer Vision and Machine Learning Background. Since no confidential data is ever leaving the Institute's network or their associated cloud service, the privacy/security/regulatory issues mentioned above can be addressed, while the model can be trained from the collaborative sharing of the learnings extracted from the data in each locality.

In most cases, the distributed model and the training process across all the separated localities can be managed by an off-site server, such as one administered by a third party that is intending to provide the resultant machine learning model as a product or service, as part of a business. It may also be a server contributed by one of the data-providing localities. This server is called the master server, acting in a similar role as described in the Parameter Server technique described above, and receives updates to the model being trained from all the other localities; each other locality is called a ‘slave’ server or node, and its purpose is to contribute to the overall trained model only from its data set that is stored locally in its own locality. A full model across all localities can thus be trained without sensitive data ever leaving an individual locality.

However whilst this the use of traditional distributed training is in-principal possible, in the absence of the privacy issues described above, traditional distributed training suffers from some important restrictions and conditions that make it inconvenient, expensive, or in some cases unusable to train a model across real-life distributed data localities, particularly when applied to confidential datasets.

Firstly, traditional distributed training requires that all servers (nodes) each have access to all the data.

The normal use-case for distributed training is not confidential data, but for facilitating training across multiple machines where each machine has access to the full data set. Each slave node therefore trains on a portion of the data set, but each node also understands how the data set has been apportioned evenly among the nodes (their file names, and how to access them). In order to be able to handle confidential data that has explicitly not been shared among the nodes, traditional distributed training must be modified so that the total number of images and their file names are available to each node (i.e., the metadata associated with the data) but not the data itself, or any confidential metadata.

Secondly, traditional distributed training requires data sets in all localities to be equally balanced.

Distributed training expects that the known full data set be equally apportioned across the localities. That is, each node has the same number of data points (e.g., medical images, as an example) as each other. This is to ensure that each node is sending weight updates to the model pertaining to the same epoch, so that a smaller data set is not over-sampled (and thus biases the model heavily on the smaller data sets). This is not typically a trivial process.

Thirdly, traditional distributed training requires the full model to be shared after each batch.

This restriction is related to cost-efficiency and practicality and relates to the fact that distributed training requires that the current state of the machine learning model must be shared by each slave node to the master after each batch. Depending on the data set, the batch size may be as small as 4-8 data points, and in cases where many thousands, tens of thousands or more data points are required to train the model even for a single epoch, and as each model can potentially be in excess of multiple gigabytes in file size, the network traffic costs can be significant.

Further, standard distributed training scales poorly with geographic distance and therefore increases the overall cost and turn-around time of training a model by up to two orders of magnitude. This problem is further exacerbated by the increase in training dataset size as more regions and data sources are connected, and therefore impacts on the time taken to train a sufficiently optimized model.

There is thus a need to provide improved methods and systems for performing distributed training of AI/machine learning models, or to at least provide a useful alternative to existing systems.

### BRIEF SUMMARY

According to a first aspect, there is provided a method for training an Artificial Intelligence (AI) model on a distributed dataset comprising a plurality of nodes, wherein, and each node comprising a node dataset and the nodes are prevented from accessing other node datasets, comprising:

generating a plurality of trained Teacher models, wherein each Teacher model is a deep neural network model which is locally trained at a node on the node dataset;

moving the plurality of trained Teacher models to a central node, wherein moving a Teacher model comprises transmitting a set of weights representing the Teacher model to the central node;

training a Student model using the plurality of trained Teacher models and a transfer dataset using knowledge distillation.

In one form, prior to moving the plurality of trained Teacher models to a central node, a compliance check is performed on each trained teacher note to check that the model does not contain private data from the node it was trained at.

In one form, the transfer dataset is an agreed-upon transfer data drawn from the plurality of node datasets, a distributed dataset comprised of a plurality of node transfer datasets, wherein node transfer dataset is local to a node or a mixture of agreed-upon transfer data drawn from the plurality of node datasets, and a plurality of node transfer datasets, wherein node local transfer dataset is local to a node.

In one form, the nodes exist across separate, geographically isolated localities.

In one form, the step of training the Student model comprises:

training the Student model using the plurality of trained Teacher models at each of the nodes using the node dataset.

In one form, prior to training the Student model using the plurality of trained Teacher models, the method further comprises:

forming a single training cluster for training the Student model by establishing a plurality of inter-region peering connections between each of the nodes, and wherein the transfer dataset comprises each of the node datasets.

In a further form, after training the Student model at each of the nodes, the Student model is sent to a master node, and copies of the Student model are sent to each of the nodes and assigned as worker nodes, and the master node collects and averages the weights of all worker nodes after each batch to update the Student model.

In one form, prior to sending the Student model to the master node a compliance check is performed on the Student model to check that the model does not contain private data from the node it was trained at.

In one form, the step of training the Student model comprises:

training a plurality of Student models, wherein each Student model is a Teacher model at a first node which is trained by a plurality of Teacher models at other nodes by moving the Student model to another node and training the Student model using the Teacher model at the node using the node dataset, and once the plurality of Student models are each trained, an ensemble model is generated from the plurality of trained Student models.

In one form, prior to training a plurality of Student models, the method further comprises:

forming a single training cluster for training the Student model by establishing a plurality of inter-region peering connections between each of the nodes.

In one form, prior to moving the Student model to another node a compliance check is performed on the Student model to check that the model does not contain private data from the node it was trained at.

In a further form, each Student model is trained after it has been trained at a predetermined threshold number of nodes, or each Student model is trained after it has been trained on a predetermined quantity of data at at least a threshold number of nodes, or each Student model is trained after it has been trained at each of the plurality of nodes.

In one form the ensemble model is obtained using an Average Voting method, a weighted averaging method, using a Mixture of Experts Layers (learned weighting) or using a distillation method, wherein a final model is distilled from the plurality of student models.

In one form the method further comprises using weighting to adjust a distillation loss function to compensate for differences in the number of data points at each node. In a further form the distillation loss function has the form:

Loss(x,y)=CrossEntropyLoss(S(x),y)+D(S(x),T(x))

where CrossEntropyLoss is a loss function, x represents a batch of training data to be minimized, y is the target (ground truth values) associated with each element of the batch x, and S(x) and T(x) are the distributions obtained from the Student and Teacher models, and D is a divergence metric.

In one form, an epoch comprises a full training pass of each node dataset, and during each epoch, each worker samples a subset of the available sample dataset, wherein the subset size is based a size of the smallest dataset, and the number of epochs is increased based on the ratio of a size of the largest dataset to the size of the smallest dataset.

In one form, the plurality of nodes are separated into k clusters, and the method as defined in the first aspect is performed separately in each cluster to generate k cluster models, wherein each cluster model is stored at a cluster representative node, and the method of the first aspect is performed on the k cluster representative nodes, wherein the plurality of nodes comprises the k cluster representative nodes. In a further form, one or more additional layers of nodes are created and each lower layer is generated by separating the cluster representative nodes in the previous layer into j clusters where j is less than the number of cluster representative nodes in the previous layer, and then the method as defined in any one of claims 1 to 25 is performed separately in each cluster to generate j cluster models, wherein each cluster model is stored at a cluster representative node, and the method as claimed in any one of claims 1 to 25 is performed on the j cluster representative nodes, wherein the plurality of nodes comprises the j cluster representative nodes.

In a further form, wherein each node dataset is medical dataset comprising one or more medical images or medical diagnostic datasets. In a further form the trained AI model is deployed.

According to a second aspect, there is provided a cloud based computation system configured to implement the method of the first aspect. This may comprise:

a plurality of local computational nodes, each local computational node comprising one or more processors, one or more memories, one or more network interfaces, and one or more storage devices storing a local node dataset wherein access to the local node dataset is limited to the respective local computational node; and

at least one cloud based central node comprising one or more processors, one or more memories, one or more network interfaces, and one or more storage devices, wherein the at least one cloud based central node is in communication with the plurality of local nodes,

wherein each of the plurality of local computational nodes and the least one cloud based central node are configured to implement the method of the first aspect to train an Artificial Intelligence (AI) model on a distributed dataset formed of the local node datasets.

In one form, the one or more of the plurality of local computational nodes are cloud based computational nodes.

In one form, the system is configured to automatically provision the required hardware and software defined networking functionality at at least one of the cloud based computational nodes. In a further form the system further comprises a cloud provisioning module and a distribution service, wherein the cloud provisioning module is configured to search available server configurations for each of a plurality of cloud services providers, wherein each cloud service provider has a plurality of servers in an associated region, and the cloud provisioning module is configured to apportion a group of servers from one or more of plurality of cloud service providers with tags and metadata to allow a group to be managed, wherein the number of servers in a group is based on number of node locations within a region associated with a cloud service providers, and the distribution service is configured to send a model configuration to a group of servers to begin training a model, and on completion of model training, the provisioning module is configured to shut down the group of servers.

In one form, each node dataset is medical dataset comprising a plurality of medical images and/or medical related test data for performing medical assessments in relation to a patient, and the AI model is trained to classify a new medical image or medical dataset.

According to a third aspect, there is provided a cloud based computation system for training an Artificial Intelligence (AI) model on a distributed dataset comprising:

at least one cloud based central node comprising one or more processors, one or more memories, one or more network interfaces, and one or more storage devices, wherein the at least one cloud based central node is in communication with a plurality of local computational nodes where each local computational nodes stores a local node dataset wherein access to the local node dataset is limited to the respective computational node, and the least one cloud based central node are configured to implement the method of the first aspect to train an Artificial Intelligence (AI) model on a distributed dataset formed of the local node datasets.

According to a fourth aspect, there is provided a method for generating an AI based assessment from one or more images or datasets, comprising:

generating, in a cloud based computational system, an Artificial Intelligence (AI) model configured to generate an AI based assessment from one or more images or datasets according to the method of the first aspect;

receiving, from a user via a user interface of the computational system, one or more images or datasets;

providing the one or more images or datasets to the AI Model to obtain a result or classification by the AI model; and

sending the result or classification to the user, via the user interface.

According to a fifth aspect, there is provided a method for obtaining an AI based assessment from one or more images or datasets, comprising:

uploading, via a user interface, one or more images or datasets to a cloud based Artificial Intelligence (AI) model configured to generate an AI based assessment wherein the AI model is generated according to the method of the first aspect;

receiving the assessment from the cloud based AI model via the user interface.

According to a sixth aspect, there is provided a cloud based computation system for generating an AI based assessment from one or more images or datasets, the cloud based computation system comprising:

one or more computational servers comprising one or more processors and one or more memories configured to store an Artificial Intelligence (AI) model configured to generate an assessment from one or more images or datasets wherein the AI model is generated according to the method of the first aspect, and wherein the one or more computational servers are configured to:

receive, from a user via a user interface of the computational system, one or more images or datasets;

provide the one or more images or datasets to the AI Model to obtain an assessment; and

send the assessment to the user, via the user interface.

According to a seventh aspect, there is provided a computation system for generating an AI based assessment from one or more images or datasets, the computation system comprising at least one processor, and at least one memory comprising instructions to configure the at least one processor to:

upload, via a user interface, an image or dataset to a cloud based Artificial Intelligence (AI) model wherein the AI model is generated according to the method of the first aspect;

receive the assessment from the cloud based AI model via the user interface.

In the fourth to seventh aspects, the one or more image or datasets may be medical images and medical datasets and the assessment is a medical assessment of a medical condition, diagnosis or treatment.

### BRIEF DESCRIPTION OF THE SEVERAL VIEWS OF THE DRAWINGS

Embodiments of the present disclosure will be discussed with reference to the accompanying drawings wherein:

FIG. 1A is a schematic diagram of a system for decentralized training of an Artificial Intelligence (or machine learning) model according to an embodiment;

FIG. 1B is a schematic block diagram of a cloud based computation system configured to computationally generate and use an AI model according to an embodiment;

FIG. 1C is schematic architecture diagram of cloud based computation system configured to generate and use an AI model according to an embodiment;

FIG. 1D is a schematic flowchart of a model training process on a training server according to an embodiment;

FIG. 1E is schematic architecture diagram of a deep learning method, including convolutional layers, which transform the input image to a prediction, after training, according to an embodiment;

FIG. 2 is a flowchart of a method for decentralized training of an AI model according to an embodiment;

FIG. 3 is a schematic diagram of multi-level process for performing decentralized training of an AI model according to an embodiment;

FIG. 4A is bar chart of the model results (balanced accuracy) for a first case study using a 5 node cluster for a baseline model, and two decentralized models on a clean dataset according to an embodiment;

FIG. 4B is bar chart of the model results (balanced accuracy) for the first case study using a 5 node cluster for a baseline model, and two decentralized models on a noisy dataset according to an embodiment;

FIG. 5 is a bar chart of the model results (balanced accuracy) for the first case study for several decentralized model using different transfer data set scenarios according to an embodiment;

FIG. 6A is bar chart of the model results (balanced accuracy) against a validation dataset for a second case study using 15 nodes in a single cluster for a baseline model, and four decentralized models on a noisy dataset and using a different number of epochs at each node according to an embodiment;

FIG. 6B is bar chart of the model results (balanced accuracy) against a test dataset for the second case study using 15 nodes in a single cluster for a baseline model, and four decentralized models on a noisy dataset and using a different number of epochs at each node according to an embodiment;

FIG. 7A is bar chart of the model results (balanced accuracy) against a validation dataset for a third case study using 15 nodes divided into 3 clusters for a baseline model, and six decentralized models on a noisy dataset and using a different number of epochs at each node according to an embodiment;

FIG. 7B is bar chart of the model results (balanced accuracy) against a test dataset for the third case study using 15 nodes divided into 3 clusters for a baseline model, and six decentralized models on a noisy dataset and using a different number of epochs at each node according to an embodiment;

FIG. 8A is bar chart of the model results (balanced accuracy) against a validation dataset for the third case study using 15 nodes divided into 3 clusters for a baseline model, and two decentralized models on a noisy dataset and using a different number of epochs at each node and visiting each node 5 times according to an embodiment; and

FIG. 8B is bar chart of the model results (balanced accuracy) against a test dataset for the third case study using 15 nodes divided into 3 clusters for a baseline model, and two decentralized models on a noisy dataset and using a different number of epochs at each node and visiting each node 5 times according to an embodiment.

In the following description, like reference characters designate like or corresponding parts throughout the figures.

### DETAILED DESCRIPTION

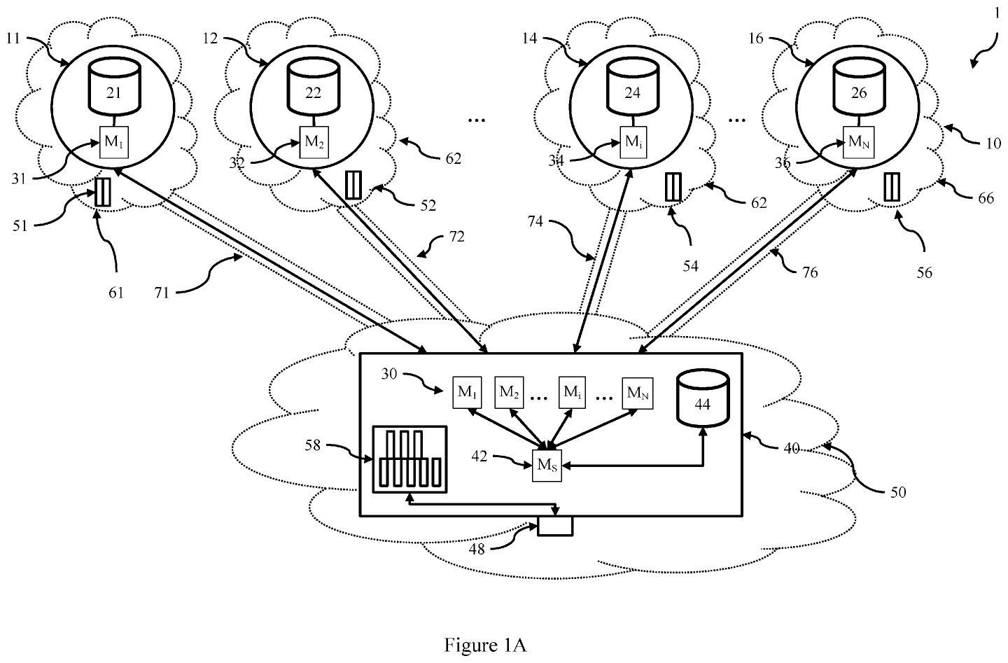

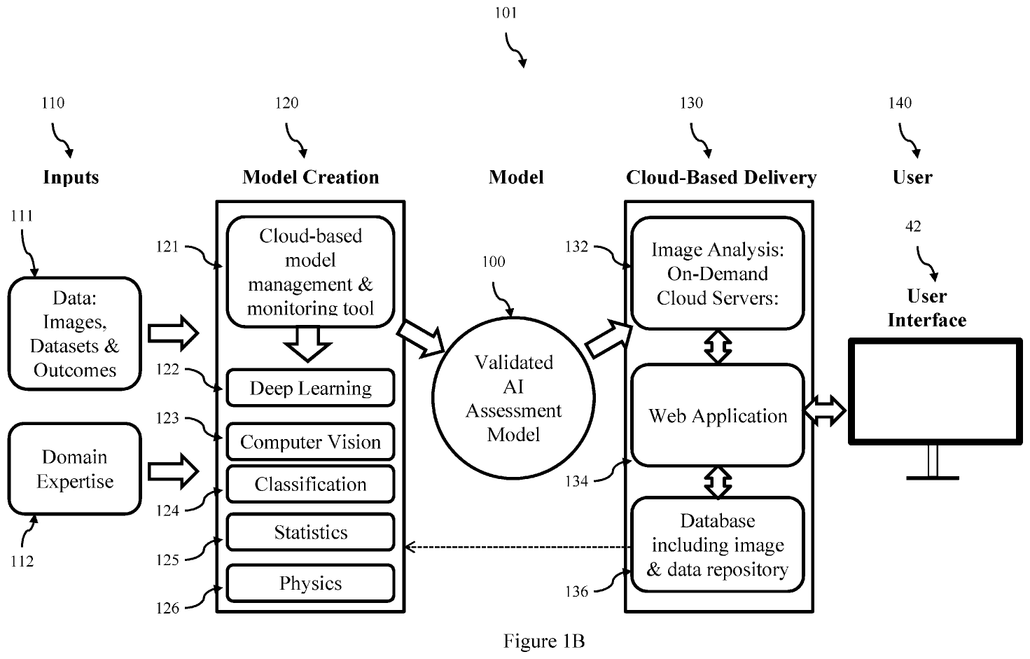

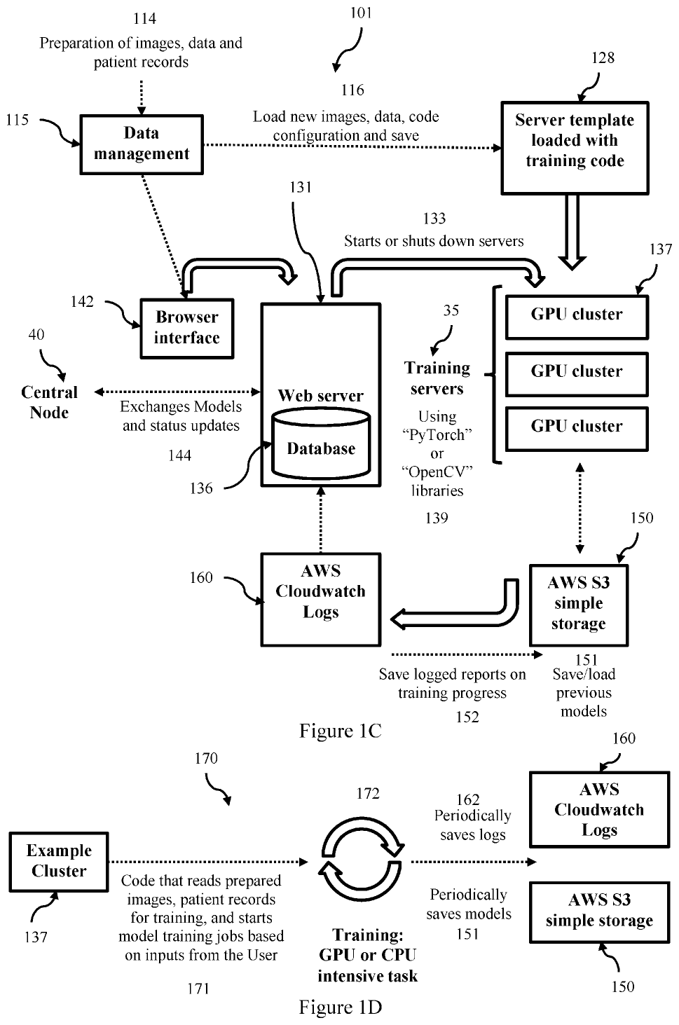

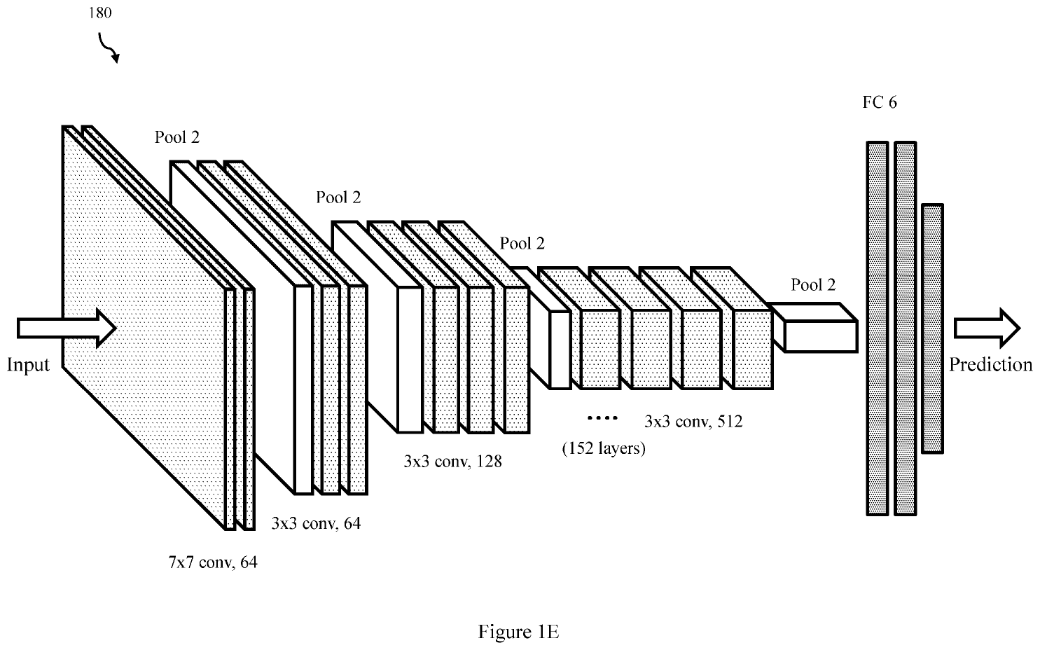

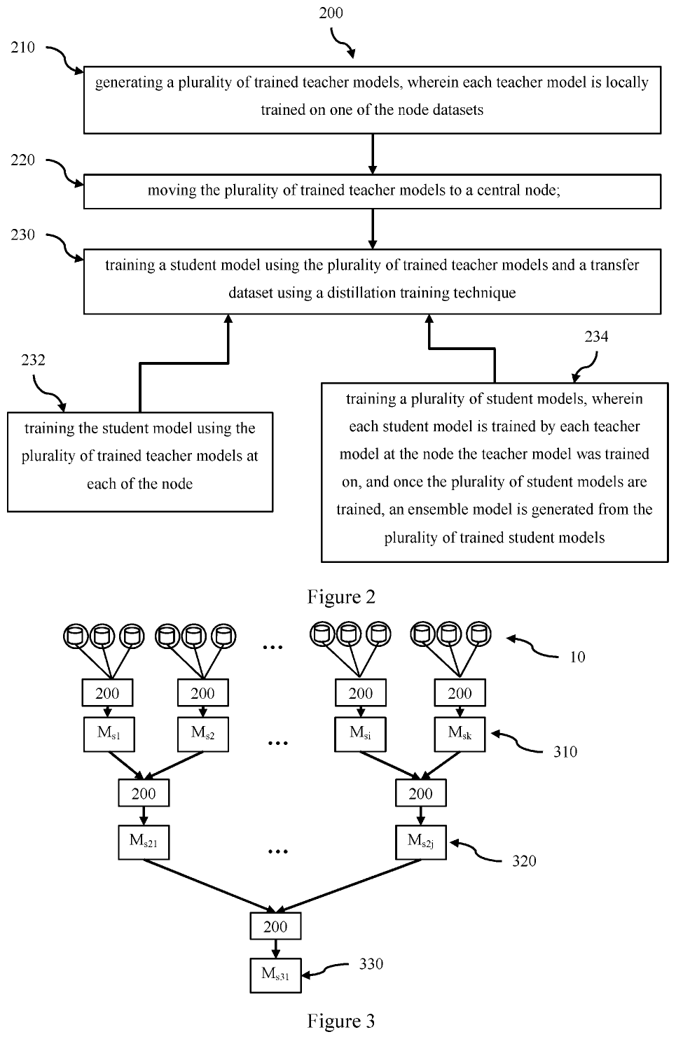

Referring now to FIG. 1A, there is shown a schematic diagram of a system 1 for decentralized training of an Artificial Intelligence (or machine learning) model according to an embodiment. FIG. 2 is a flowchart 200 of a method for decentralized training of an AI model on a distributed dataset according to an embodiment.

FIG. 1A shows a distributed system comprised of a plurality (M) of nodes 10. Each individual node 11, 12, 14, 16 each comprises a local dataset 21, 22, 24, 26. The nodes 10 are operationally isolated, so that the local dataset in each node is prevented from leaving the node, or being remotely accessed by another node or process. This isolation may physical, such as geographic isolation, software isolation, such as through the use of firewalls and software based security measures, or some combination of the two. The nodes may be geographically distributed over a single country or continent or over multiple countries or continents. The nodes 10 may be cloud based nodes, hosted in local clouds 61, 62, 64, and 66, comprising local cloud computing resources 51, 52, 54, 56 such as processors, memories and networking interfaces which can be configured to run software applications within the local cloud, and to exchange information with external resources and processes as required (or authorized).

In one embodiment the local dataset is be a medical dataset for performing a medical assessment where sharing of the dataset with third parties is not allowed. This may include medical image data and medical related testing data, including screening tests, diagnostic tests and other data for assessing a medical condition, making a diagnosis, developing a treatment plan or making a medical related decision (e.g., which embryo to implant in an IVF procedure). The medical dataset may also include associated metadata including patient data, data relating to the image or test (e.g., equipment configuration and measurements) and outcomes. A patient record may comprise one or more medical images and/or testing data associated with a patient, and outcomes. The medical image data may be a set of images related to a patient, including video data, such as x-ray, ultrasound, MRI, camera, and microscopic images. These may be of a body part or section of a body, biopsy, one or more cells, or image of a diagnostic, screening or assessment test or equipment including multi-well plates, microarrays, etc. Similarly the test data may be a set of diagnostic or screening tests and may be a complex datasets including a time series of results of specific biomarkers, metabolic measurements such as a blood panel or complete or full blood count, genomic data, sequencing data, proteomics data, metabolics data, etc. In these embodiments the AI model is trained to analyze or classify a medical image or diagnostic test data, such that in use the trained AI model can be deployed to analyze or classify a new medical image or new diagnostic test data from a patient to diagnose a specific disease or medical condition. This may include a range of specific cancers, embryo viability and fertility conditions, thoracic conditions such as pneumonia (e.g., using chest x-rays), blood and metabolic disorders, etc. The medical dataset may be used for assessing or diagnosing a range of medical conditions and diseases or to assist in medical decision making and treatments. For example images of embryos taken in the days after in-vitro-fertilization (IVF) may be used for assessing embryo viability to assist in embryo selection, Chest X-rays and Chest CT scans may be used for identifying pneumonia and other lung conditions. X-ray, CT and MRI scans maybe used to diagnosing solid cancers. Retinal images can be used for assessing glaucoma and other eye diseases. Blood panels, pathology tests, point of care tests, antibody, DNA/SNP/protein arrays, genomic sequencing, proteomics, metabolomics datasets may be used for identifying medical conditions and diseases, blood disorders, metabolic disorders, biomarkers, classification of disease subtypes, identification of treatments, identification of disease and lifestyle risk factors, etc. The training dataset is created to enable training of the AI model and may contain labels used to train the data, or the AI model may learn to classify the data during training. In other embodiments the data may relate to non-healthcare applications, such as security and defense applications where privacy, defense, or commercial concerns prevent the sharing of data between difference sources, but where it would be desirable to leverage the power of a large dataset. This data may be image surveillance data (e.g., security cameras), location data, purchasing data, etc.

As noted above the local nodes and central node may be cloud based computational systems. FIG. 1B is a schematic architecture diagram of cloud based computation system 100 configured to generate (train) and use (including deploy) a trained AI model 100 according to an embodiment. The training and use of an AI model is further illustrated in FIGS. 1C and 1D, with FIG. 1C illustrating the cloud architecture of a node 101 which may be under control of the model monitor 121 which coordinates generation of the AI model at the central node 40. Embodiments of this cloud architecture may be used for each local node 10 as well as the central node 40. The nodes may also be hosted in a commercial cloud environment (e.g., Amazon Web Services, Microsoft Azure, Google Cloud platform, etc.), a private cloud, or use a local server farm with a similar configuration to that illustrated in FIGS. 1C and 1D.

The model monitor 121 allows a user (administrator) to provide images and datasets, along with associated and metadata 114 to a data management platform which includes a data repository 115 (local to the node). A data preparation step may be performed, for example to move the images to specific folder, and to rename and perform pre-processing on the images such as objection detection, segmentation, alpha channel removal, padding, cropping/localizing, normalizing, scaling, etc. Feature descriptors may also be calculated, and augmented images generated in advance. Similarly datasets may be parsed and reformatted into a standard format/table, cleaned and summarized. However additional pre-processing including augmentation may also be performed during training (i.e., on the fly). Images and dataset may also undergo quality assessment, to allow rejection of clearly poor images or erroneous data and allow capture of replacement images or data. Similarly patient records or other clinical (or other) data is processed (prepared) for example to identify an outcome measure which is linked or associated with each image/dataset to enable use in training the AI models and/or in assessment. The prepared data is loaded 116 onto a cloud provider (e.g., AWS) template server 128 with the most recent version of the training algorithms (which may be provided by the central node 40). The template server is saved, and multiple copies made across a range of training server clusters 137, which may be CPU, GPU, ASIC, FPGA, or TPU (Tensor Processing Unit)-based, which form (local) training servers 135. The local model monitor web server 131 then applies for a training server 137 from a plurality of cloud based training servers 135 for each job submitted by the model monitor in the central node 140. Each training server 135 runs the pre-prepared code (from template server 128) for training an AI model, using a library such as Pytorch, Tensorflow or equivalent, and may use a computer vision library such as OpenCV. PyTorch and OpenCV are open-source libraries with low-level commands for constructing CV machine learning models.

The training servers 137 manage the training process. This may include may dividing the images and data in to training, validation, and blind validation sets, for example using a random allocation process. Further during a training-validation cycle the training servers 137 may also randomize the set of images at the start of the cycle so that each cycle a different subset of images are analyzed, or are analyzed in a different ordering. If pre-processing was not performed earlier or was incomplete (e.g., during data management) then additional pre-processing may be performed including object detection, segmentation and generation of masked data sets, calculation/estimation of CV feature descriptors, and generating data augmentations. Pre-processing may also include padding, normalizing, etc., as required. Pre-processing may be performed prior to training, during training, or some combination (i.e., distributed pre-processing). The number of training servers 135 being run can be managed from the browser interface. As the training progresses, logging information about the status of the training is recorded 162 onto a distributed logging service such as Cloudwatch 160. Accuracy information is also parsed out of the logs and saved into a relational database 36. The models are also periodically saved 151 to a data storage (e.g., AWS Simple Storage Service (S3) or similar cloud storage service, or local storage) 150 so they can be retrieved and loaded at a later date (for example to restart in case of an error or other stoppage). The model monitor/central node 140 exchanges models, training instructions and status updates with the local model monitor server 131 over a communication link. The status updates may provide the status of the training servers such as when their jobs are complete, or an error is encountered.

Within each training cluster 137, a number of processes take place. Once a cluster is started via the web server 131, a script is automatically run, which reads the prepared images and patient records, and begins the specific Pytorch/OpenCV training code requested 171. The input parameters for the model training 128 are supplied by the model monitor 121 at the central node 140. The training process 72 is then initiated for the requested model parameters, and can be a lengthy and intensive task. Therefore, so as not to lose progress while the training is in progress, the logs are periodically saved 162 to the logging (e.g., AWS Cloudwatch) service 160 and the current version of the model (while training) is saved 151 to the data (e.g., S3) storage service 151 for later retrieval and use. With access to a range of trained AI models on the data storage service, multiple models can be combined together for example using ensemble, distillation or similar approaches in order to incorporate a range of deep learning models (e.g., PyTorch) and/or targeted computer vision models (e.g., OpenCV) to generate a robust AI model 100 which is provided to the cloud based delivery platform 130.

Once a trained model is generated it may be deployed for use. The cloud-based delivery platform 130 system then allows users 110 to drag and drop images or datasets directly onto the web application 134, which prepares the image/dataset and passes this to the trained/validated AI model 100 to obtain a classification/result which is immediately returned in a report. The web application 134 also allows clinics to store data such as images and patient information in database 136, create a variety of reports on the data, create audit reports on the usage of the tool for their organization, group or specific users, as well as billing and user accounts (e.g., create users, delete users, reset passwords, change access levels, etc.). The cloud-based delivery platform 130 also enables product admin to access the system to create new customer accounts and users, reset passwords, as well as access to customer/user accounts (including data and screens) to facilitate technical support.

The training process comprises pre-processing data, for example alpha channel stripping, padding/bolstering, normalizing, threshold, object detection/cropping, extraction of geometrical properties, zooming, segmenting, annotation, resizing/scaling, and tensor conversion. The data may also be labelled and cleaned. Once the data is suitably pre-processed it can then be used to train one or more AI models. Computer vision image descriptors may also be calculated on images. These descriptors may encode qualities such as pixel variation, gray level, and roughness of texture, fixed corner points or orientation of image gradients, which are implemented in the OpenCV or similar libraries. By selection on such feature to search for in each image, a model can be built by finding which arrangement of the features is a good indicator for the outcome class. These may be pre-calculated or calculated during model generation/training.

Training is performed using randomized datasets. Sets of complex image data, can suffer from uneven distribution, especially if the data set is smaller than around 10,000 images, where exemplars of key viable or non-viable embryos are not distributed evenly through the set. Therefore, several (e.g., 20) randomizations of the data are considered at one time, and then split into the training, validation and blind test subsets defined below. All randomizations are used for a single training example, to gauge which exhibits the best distribution for training. As a corollary, it is also beneficial to ensure that the ratio between the number of different classes is the same across every subset to ensure even distribution of images/data across test and training sets to improve performance. Training further comprises performing a plurality of training-validation cycles. In each train-validate cycle each randomization of the total useable dataset is split into typically 3 separate datasets known as the training, validation and blind validation datasets. In some variants more than 3 could be used, for example the validation and blind validation datasets could be stratified into multiple sub test sets of varying difficulty.

The first set is the training dataset and comprises at least 60% and preferably 70-80% of images. These images are used by deep learning models and computer vision models to create the AI assessment/classification model. The second set is the Validation dataset, which is typically around (or at least) 10% of images: This dataset is used to validate or test the accuracy of the model created using the training dataset. Even though these images/data are independent of the training dataset used to create the model, the validation dataset still has a small positive bias in accuracy because it is used to monitor and optimize the progress of the model training. Hence, training tends to be targeted towards models that maximize the accuracy of this particular validation dataset, which may not necessarily be the best model when applied more generally to other embryo images. The third dataset is the Blind validation dataset which is typically around 10-20% of the images. To address the positive bias with the validation dataset described above, a third blind validation dataset is used to conduct a final unbiased accuracy assessment of the final model. This validation occurs at the end of the modelling and validation process, when a final model has been created and selected. It is important to ensure that the final model's accuracy is relatively consistent with the validation dataset to ensure that the model is generalizable to all embryos images. The accuracy of the validation dataset will likely be higher than the blind validation dataset for the reasons discussed above. Results of the blind validation dataset are a more reliable measure of the accuracy of the model.

The architecture of a DNN is constrained by the size of images/data as input, the hidden layers, which have dimensions of the tensors describing the DNN, and a linear classifier, with the number of class labels as output. Most architectures employ a number of down-sampling ratios, with small (3×3 pixel) filters to capture notion of left/right, up-down and center. Stacks of a) Convolutional 2d layers, b) Rectified Linear Units (ReLU), and c) Max Pooling layers allow the number of parameters through the DNN to remain tractable, while allowing the filters to pass over the high level (topological) features of an image, mapping them onto the intermediate and finally microscopic features embedded in the image. The top layer typically includes one or more fully-connected neural network layers, which act as a classifier, similar to SVM. Typically, a Softmax layer is used to normalize the resulting tensor as containing probabilities after the fully connected classifier. Therefore, the output of the model is a list of probabilities that the image/data is either in a class or not for each class.

FIG. 1E is schematic architecture diagram of a deep learning method, including convolutional layers, which transform the input image to a prediction, after training, according to an embodiment. FIG. 1E shows a series of layers based on a RESNET 152 architecture according to an embodiment. The components are annotated as follows. “CONV” indicates a convolutional 2D layer, which computes cross-correlations of the input from the layer below. Each element or neuron within the convolutional layer processes the input from its receptive field only, e.g., 3×3 or 7×7 pixels. This reduces the number of learnable parameters required to describe the layer, and allows deeper neural networks to be formed than those constructed from fully-connected layers where every neuron is connected to every other neuron in the subsequent layer, which is highly memory intensive and prone to overfitting. Convolutional layers are also spatial translation invariant, which is useful for processing images where the subject matter cannot be guaranteed to be precisely centered. “POOL” refers the max pooling layers, which is a down-sampling method whereby only representative neuron weights are selected within a given region, to reduce the complexity of the network and also reduce overfitting. For example, for weights within a 4×4 square region of a convolutional layer, the maximum value of each 2×2 corner block is computed, and these representative values are then used to reduce the size of the square region to 2×2 in dimension. RELU indicates the use of rectified linear units, which act as a nonlinear activation function. As a common example, the ramp function takes the following form for an input x from a given neuron, and is analogous to the activation of neurons in biology f(x)=max(0, x). The final layers at the end of the network, after the input has passed through all of the convolutional layers, is typically a fully connected (FC) layer, which acts as a classifier. This layer takes the final input and outputs an array of the same number of dimensions as the classification categories. For two categories, the final layer will output an array of length 2, which indicates the proportion that the input image/data contains features that align with each category respectively. A final softmax layer is often added, which transforms the final numbers in the output array to percentages that fit between 0 and 1, and both together add up to a total of 1, so that the final output can be interpreted as a confidence limit for the image to be classified in one of the categories.

As discussed above both computer vision and deep learning methods are trained using a plurality of Train-Validate Cycles on pre-processed data. The Train-Validate cycle follows the following framework: The training data is pre-processed and split into batches (the number of data in each batch is a free model parameter but controls how fast and how stably the algorithm learns). Augmentation may be performed prior to splitting or during training. After each batch, the weights of the network are adjusted, and the running total accuracy so far is assessed. In some embodiment weights are updated during the batch for example using gradient accumulation. When all images have been assessed 1 Epoch has been carried out, the training set is shuffled (i.e., a new randomization with the set is obtained), and the training starts again from the top, for the next epoch.

During training a number of epochs may be run, depending on the size of the data set, the complexity of the data and the complexity of the model being trained. An optimal number of epochs is typically in the range of 2 to 100, but may be more depending on the specific case. After each epoch, the model is run on the validation set, without any training taking place, to provide a measure of the progress in how accurate the model is, and to guide the user whether more epochs should be run, or if more epochs will result in overtraining. The validation set guides the choice of the overall model parameters, or hyperparameters, and is therefore not a truly blind set. However, it is important that the distribution of images of the validation set is very similar to the ultimate blind test set that will be run after training. Pre-training, or transfer learning, where a previously trained model is used as the starting point to train a new model may be used. For non pre-trained models, or new layers added after pre-training such as the classifier, the weights need to be initialized. The initialization method can make a difference to the success of the training. All weights set to 0 or 1, for example, will perform very poorly. A uniform arrangement of random numbers, or a Gaussian distribution of random numbers, also represent commonly used options. These are also often combined with a normalization method, such as Xavier or Kaiming algorithms. This addresses an issue where nodes in the neural network can become ‘trapped’ in a certain state, by becoming saturated (close to 1), or dead (close to 0), where it is difficult to measure in which direction to adjust the weights associated with that particular neuron. This is especially prevalent when introducing a hyperbolic-tangent or a sigmoid function, and is addressed by the Xavier initialization.

In deep learning, a range of free parameters is used to optimize the model training on the validation set. One of the key parameters is the learning rate, which determines by how much the underlying neuron weights are adjusted after each batch. When training a selection model, overtraining, or overfitting the data should be avoided. This happens when the model contains too many parameters to fit, and essentially ‘memorizes’ the data, trading generalizability for accuracy on the training or validation sets. This is to be avoided, since the generalizability is the true measure of whether the model has correctly identified true underlying parameters that indicate embryo health, among the noise of the data, and not compromised this in order to fit the training set perfectly.

During the Validation and Test phases, success rates can sometimes drop suddenly due to overfitting during the Training phase. This can be ameliorated through a variety of tactics, including slowed or decaying learning rates (e.g., halve the learning rate every n epochs) or the use of CosineAnnealling, incorporating the aforementioned methods of tensor initialization or pre-training, and the addition of noise, such as Dropout layers, or Batch Normalization. Batch Normalization is used to counteract vanishing or exploding gradients which improves the stability of training large models resulting in improved generalization. Dropout regularization effectively simplifies the network by introducing a random chance to set all incoming weights zero within a rectifier's receptive range. By introducing noise, it effectively ensures the remaining rectifiers are correctly fitting to the representation of the data, without relying on over-specialization. This allows the DNN to generalize more effectively and become less sensitive to specific values of network weights. Similarly, Batch Normalization improves training stability of very deep neural networks, which allow s for faster learning and better generalization by shifting the input weights to zero mean and unit variance as a precursor to the rectification stage.

In performing deep learning, the methodology for altering the neuron weights to achieve an acceptable classification includes the need to specify an optimization protocol. That is, for a given definition of ‘accuracy’ or ‘loss’ (discussed below) exactly how much the weights should be adjusted, and how the value of the learning rate should be used, has a number of techniques that need to be specified. Suitable optimization techniques include Stochastic Gradient Descent (SGD) with momentum (and/or Nesterov accelerated gradients), Adaptive Gradient with Delta (Adadelta), Adaptive Moment Estimation (Adam), Root-Mean-Square Propagation (RMSProp), and Limited-Memory Broyden-Fletcher-Goldfarb-Shanno (L-BFGS) Algorithm. Of these, SGD based techniques generally outperformed other optimization techniques. For example learning rates for training an AI model on phase contrast microscope images of human embryos were between 0.01 to 0.0001. However this is one example and the learning rate will depend upon batch size, which is dependent upon hardware capacity. For example larger GPUs allow larger batch sizes and higher learning rates. Once a range of models have been trained, these may be combined using ensemble or distillation techniques to generate a final model which can then be deployed.

In a first embodiment, which we will refer to as simple distillation, an AI model is generated by generating a plurality of trained teacher models 210 where each Teacher model M₁, M₂, . . . , Mᵢ, . . . , M_{N}, is locally trained on one of the node datasets (i.e., one model per node). Once each of the teacher models 30 are trained, they are moved 220 to a central node 40. A student model 42 Mₛ is then trained using the plurality of trained Teacher models 30 on a transfer dataset 44 using a distillation training technique/method 230. The trained Student model Mₛ is the output AI model which is stored and used to generate outcomes when presented with new data.

The initial local training of the Teacher models at the nodes may be performed using any suitable AI or machine learning training architecture. The teacher models can be of any deep neural network (DNN) architecture, and can even be completely different architectures from each other. For example, the AI architecture may be based on RESNET, DENSET, INCEPTION NET, EFFICIENT NET, etc., neural network architectures. AI and machine learning libraries such as Torch/Pytorch, Tensorflow, Keras, MXNet or equivalent, may be used as well as computer vision library such as OpenCV, Scikit-image and Scikit-learn. These libraries are open-source libraries with low-level commands for constructing machine learning models.

Each model that is a Student model, Teacher model, or described as a specialist (a model trained on a single locality only) or a generalist model (the full model taking into account representatives from multiple localities) will each refer to a deep neural network. Each network is fully characterized by the ways its component ‘neurons’ are organized and connected together (the architecture of [0045]) and the values of the weight parameters themselves. The neurons themselves are simply elements of a multi-dimensional array (mathematically, a ‘tensor’), with a specific activation function, such as the Rectified Linear Unit (ReLU), which is designed to mimic biological neural activation, after a matrix multiplication has been applied to each level of the array. The network weights themselves are the numerical representation for each network's connection among neurons. A network/model is trained/learned by adjusting its weights to minimize the cost of a particular objective function (such as the mean squared error between network's outputs and the actual class labels of samples regarding a classification problem). After or during the training process, the network's weights are changed, and one can checkpoint/save the network's weights to a file which can be stored locally or on Amazon cloud storage S3, or other cloud storage service (called checkpointed file, or trained model file). As described herein moving or transferring a model to another node describes transferring the neural network's weights, and pre-defined architecture, securely using a protocol such as https. In distillation-inspired training, Student and Teacher models, specialist and generalist models can be any kind of aforementioned networks. In the context of distillation training, terms such as ‘sending a Student model to each node,’ ‘sending a Teacher model to become a Student in another locality,’ ‘update the master node with the worker models,” etc., have the same meaning: namely transferring the network's weights from one location/machine/server to another. Given that the training process for training a neural network model is well-established, a checkpointed Student network's weight is sufficient information for a model to continue to train from, regardless of whether the model now takes the role of a Teacher in respect to the others, as is the case in the distillation learning procedure explained further below. Thus in the context of the specification copying or moving a model will refer to copying or moving at least the network weights. In some embodiments this may involve exporting or saving a checkpoint file or a model file using an appropriate function of the machine learning code/API. The checkpoint file may be a file generated by the machine learning code/library with a defined format which can be exported and then read back in (reloaded) using standard functions supplied as part of the machine learning code/API (e.g., ModelCheckpoint( ) and load_weights( )). The file format may directly sent or copied (e.g., ftp or similar protocols) or it be serialized and send using JSON, YAML or similar data transfer protocols. In some embodiments additional model metadata may be exported/saved and sent along with the network weights, such as model accuracy, number of epochs, etc., that may further characterize the model, or otherwise assist in constructing another model (e.g., a Student model) on another node/server.

The transfer dataset 44 is a dataset that is decided upfront prior to training, that it is established as data that is permitted to be used and accessed by both the Teacher and Student models during the distillation method. It may be sourced from the total original dataset intending to train on, e.g., this may be an authorized set of data obtained from among the nodes 10, for which distribution access has been granted, or it may be a new dataset collected for the purpose of training and not subject to privacy or other access restrictions which are applied to the node datasets. Alternatively, the transfer dataset can also be the dataset in each locality, i.e., there can be a different transfer dataset for each locality. The transfer dataset may be mixture of agreed upon data drawn from the plurality of node datasets and local datasets. The transfer dataset may be used to calibrate a Student model from the Teacher during the knowledge distillation process.

In some embodiments, a compliance check is performed to check that the model does not contain private data before it is shared. Table 1 presents pseudocode for an embodiment of a compliance check method which is used to check if a model capable of storing private data has done so, and must ensure that compliance is respected, so that no private data is allowed to leave the locality. This is achieved by checking whether the model has memorized specific examples or has generalized correctly, by performing a data leak check, for example, a Nearest Neighbors algorithm that can identify if examples generated from the model are similar to examples in the data set.

The output of a function check_no_data_leak(Mᵢ, Mᵢ′, Dᵢ, Dᵢ′) is TRUE if the model Mᵢ has generalized correctly, or FALSE, if it has not generalized correctly. This function must generate a data set Dᵢ′, which is the dataset that can be derived from the newly trained model Mᵢ on Dᵢ, minus (or excluding) the dataset that can be derived from the original model Mᵢ′ before training on Dᵢ (to prevent the situation where a datapoint in Dᵢ matches data that can be derived from the original model Mᵢ′ before training on Dᵢ). Dᵢ′ can be compared to the dataset Dᵢ to make sure there is no data leak.

If it is FALSE, an additional task must be performed to ensure compliance is respected. This includes:

(1) retrain Mᵢ′ from the data Dᵢ with different parameters and recheck that it cannot replicate the data, and if unsuccessful repeat N times before giving up and selecting alternative option (2), then (3) or (4); if unsuccessful, then

(2) try server Sᵢ again later (e.g., when all others in the cluster are complete) with a new and different Mᵢ′ and try (1) again one more time; if unsuccessful then either perform (3) or (4):

(3) do not allow the model to contribute to the general model (i.e., ignore it by returning Mᵢ′); or

(4) perform an encryption process on the weights, gradients, or data, in any combination, before the encrypted model potentially containing the private data is shared and aggregated (only if data policies allow encrypted sharing of private data).

TABLE 1
Pseudocode for Compliance Check Function That
Checks Mᵢ' for Data Compliance.

| | |
|---|---|
| 1 | METHOD: compliance_check( ) |
| 2 | IF model_type_that_can_store_private_data(Mᵢ) == TRUE: |
| 3 | IF check_no_data_leak(Mᵢ, Mᵢ', Dᵢ, Dᵢ') |
| 4 | RETURN Mᵢ |
| 5 | ELSE |
| 6 | RETURN Mᵢ' |
| 7 | ELSE |
| 8 | RETURN Mᵢ |

Table 2 present pseudocode for an embodiment of the Simple Distillation algorithm. This embodiment uses the compliance check method described above and shown in Table 1.

TABLE 2
Pseudocode for Simple Distillation algorithm.

| | |
|---|---|
| 1 | Simple Distillation algorithm |
| 2 | Input: N data owner (distributed data source) servers, server Sᵢ is used for i-th data owner |
| 3 | Input:Local dataset Dᵢ at Sᵢ, without external access |
| 4 | Input: Central server S_{c}, with its own local (transfer) dataset D_{c} |
| 5 | Input: Distillation loss function L |
| 6 | |
| 7 | FOR i in range[N] |
| 8 | Initialize AI teacher model Mᵢ at Sᵢ |
| 9 | Train Mᵢ with the local data Dᵢ at Sᵢ until converged |
| 10 | IF compliance_check( ) |
| 11 | Move trained Mᵢ to S_{c} |
| 12 | ENDFOR |
| 13 | |
| 14 | Initialize the AI student model M_{c} at S_{c} |
| 15 | Train M_{c} using the local transfer dataset D_{c}, using L on each teacher (Mᵢ)'s outcome or on an |
| | ensemble of all teacher (Mᵢ)s' outcomes |
| 16 | RETURN M_{c} |

In another embodiment, which we will refer to as modified decentralized distillation, the last distillation step is modified wherein training the student model Mₛ comprises training the student model Mₛ using the plurality of trained teacher models M₁, M₂, . . . , Mᵢ, . . . , M_{N}, at each of the nodes (i.e., step 230 is replaced with step 232 in FIG. 2). This is facilitated by forming a single training cluster for training the student model by establishing a plurality of inter-region peering connections 71, 72, 74 and 76 between each of the nodes 11, 12, 14, 16 and the central node 40. Thus in this embodiment the transfer dataset 44 comprises each of the node datasets 21, 22, 24, and 26, thus allowing the student model to have access to the full dataset.

For example, we have Student model S and Teacher models T₁, T₂ and T₃ where each is a distinct location, where sharing of the dataset itself is not permitted, but sharing of the neural network weights and non-confidential metadata is permitted (e.g., model architectures, de-identified file numbers, number of total data points in each region, etc.). The total number of Teachers is typically equivalent to the total number of distinct locations (e.g., clinics). The training code and the code for receiving and sending network's weight are assumed to be available at each location/machine. For simplicity, S and Tᵢ denote the model names and are also the model's weight that can be sent to other nodes. A cloud-based master or administrative server node controls the training procedure and collects the final trained model for production.

In the proposed modified decentralized distillation, first, all Teachers are trained based on their local data independently. Then S is sent to each Tᵢ locality, and performs distillation learning based on local data and locally-trained Teacher. After S is trained for several rounds, its own weights are refined based on the distillation from the local Teacher model. To further improve S, the distillation method is concluded, and then a final decentralized training stage may be performed with a small number of training iterations. This final step ensures that S has a chance to be exposed to all the data simultaneously, and mitigate any bias on a specific clinic associated with the ordering of the distillation process. The decentralized step, while exhibiting higher cost in terms of time and network transfer, only need to applied for a much smaller number of iterations or epochs, due to the fact that the distillation process has acted as an effective pretraining step. In the decentralized training stage, S becomes the master node, and copies of S: S₁, S₂ and S₃ are made, to be sent to each Tᵢ location respectively. Each Sᵢ will now act as a worker node during this stage. While performing training, the master node will collect and average the gradients/weights of all worker nodes after each batch, and thus S can be exposed to all the data allocated in different locations at the same time. Table 3 present pseudocode for an embodiment of the Modified Decentralized Distillation algorithm.

As each of the Teacher models have been trained prior to the distillation step, the student model will be faster to train and a much smaller number of iterations are needed, therefore reducing costs. Furthermore we can have access to the full dataset for a limited time, helping to improve the generalization capability of the Student model. This will be more expensive than the simple distillation case, but significantly less than training a fully decentralized model.

TABLE 3
Pseudocode for Modified Decentralized Distillation algorithm.

| | |
|---|---|
| 1 | Input: N data owner servers, server Sᵢ is used for i-th data owner |
| 2 | Input: Local dataset Dᵢ at Sᵢ, without external access |
| 3 | Input: Central server S_{c}, with its own local (transfer) dataset D_{c} |
| 4 | Input: Distillation loss function L |
| 5 | Input: R is number of times student model being sent around |
| 6 | |
| 7 | Initialize the AI student model M_{c} at S_{c} |
| 8 | FOR i in range[N] |
| 9 | Initialize AI teacher model Mᵢ at Sᵢ |
| 10 | Train Mᵢ with the local data Dᵢ at Sᵢ until converged |
| 11 | ENDFOR |
| 12 | FOR r in range[R] |
| 13 | IF compliance_check( ) |
| 14 | Move M_{c} to Sᵢ and train M_{c} using L on (Mᵢ)'s outcome, using local data Dᵢ |
| 15 | Move M_{c} back to S_{c} for validation purpose (optional step) |
| 16 | ENDFOR |
| 17 | IF compliance_check( ) |
| 18 | Move M_{c} to S_{c} |
| 19 | Make a copy of M_{c} at S_{c} and send to any Sᵢ, denoted as M_{ci} model |
| 20 | Perform standard decentralized training: |
| | M_{c} at S_{c} plays as a master node and uses the local transfer dataset D_{c} while |
| | M_{ci} at Sᵢ uses local data Dᵢ and acts as a worker node |
| 21 | RETURN M_{c} |

The above embodiment overcomes the limitations preventing data sharing between regions or institutions and which prevents collating the individual datasets into a single dataset which can be used to train a model. The above embodiment effectively configures the cloud services (or any networked server, fixed or portable device) and distributed training in such a way that it appears that each of the nodes (or localities or regions) is part of the same cluster. That is the local clouds 61, 62, 64 and 66, and central cloud 50 forms a connected cloud cluster. This involves, for example, setting up a cloud service provider using an inter-region peering connection. This is a feature that can be used to create a cluster of different cloud regions. This is often used for mirroring databases and services across regions. One may configure this such that the standard distributed training can work in a decentralized manner.

As noted above modified decentralized distillation still suffers from a performance penalty due to the need for the Student model to the shared after each batch (e.g., weights need to be sent from each location). Training deep learning models is time consuming, even when training on a single machine or node. When scaling up the system to use multiple regions, the training time for a simple model increases by a factor of 100, rather than decreases, as would be the case for Distributed Training (same region and same data set), e.g., what would take 1 hour to train, will now take 4 days. This is due to the vast difference in geographic scale between training a model on nodes in the same data center, versus training a model with nodes on the other side of the planet. This is further exacerbated by the choice of gradient update pattern (Parameter Server and Ring All-Reduce). For example, some international links are more costly in terms of network latency than others, often due to geographical distance. Furthermore, cloud service providers typically charge network traffic between data centers. The exact rate depends on which regions are being used.

However additional embodiments may incorporates a number of extra features that extend the modified decentralized distillation method to improve a) accuracy from poor weighting of data among nodes, or class imbalances, and most importantly b) time/cost efficiency.

In one embodiment, loss function weighting is used.

The process of weighting involves emphasizing the contribution of particular aspects of a set of data over others to a final outcome or result; thereby highlighting those aspects in comparison to others in the analysis. That is, rather than each variable in the data set contributing equally to the final result, some of the data is adjusted to make a greater contribution than others.

This occurs by modifying the loss function used for training in the following manner. We first select a standard cross entropy loss function used for training a machine learning model which we will refer to generically as “CrossEntropyLoss.” In one embodiment this may be the log loss or binary cross entropy loss function CrossEntropyLoss(yᵢ,ŷᵢ)=−Σ(yᵢ log(ŷᵢ)+(1−yᵢ)log(1−ŷᵢ) where y is the binary indicator of whether class label c is correct for the ith element, and ŷᵢ is the model prediction that the ith element belongs to class c. Other similar loss functions and variants may be used. If x represents a batch of training data to be minimized, y is the target (ground truth values) associated with each element of the batch x, and S(x) and T(x) are the distributions obtained from the Student and Teacher models, respectively, then we define the distillation loss function as:

Loss(x,y)=CrossEntropyLoss(S(x),y)+D(S(x),T(x)) Equation 1

The function D is a divergence metric, where a common choice in practice is, e.g., the Kullback-Leibler Divergence (although other divergence metrics may be used such as Jensen-Shannon divergence):

$${D}_{KL} ( S ( x ) , T ( x ) ) = {\sum }_{xi} S ( xi ) log \frac{S ( xi )}{T ( xi )} \qquad Equation 2$$

In another embodiment, an under-sampling approach is used to improve load balancing.

For standard distributed training methods, as the gradients need to be averaged for every batch, each of the batches need to be load balanced such that they contribute equally. Furthermore, the synchronous SGD (synchronizes gradients every batch) assumes that the partial dataset on each worker is the same size. More specifically, in either the Ring All-Reduce or Parameter Server configurations, each of the nodes or master node respectively, will block (wait for network data) until it receives the gradients from each of the workers. This means that when datasets are unbalanced in terms of the number of samples available, the training will fail as the standard algorithms assume that each worker contributes equally. However, when training in the decentralized setup, the workers will have different dataset sizes for each region.

To address this issue an under-sampling method may be used, in which for each epoch (one full training pass of the dataset) each worker only samples a subset of the available sample. The under-sampling amount is chosen to be equal to the smallest dataset available in the collection. This has the effect of equally weighting the samples from all of the different regions, which can also help with the robustness of the model. However, as some datasets in the collection will be much larger than others, the total number of iterations of the dataset needs to be increased, i.e., the number of epochs needs to be scaled by under-sampling ratio. For example, if the smallest dataset is 100 samples, and the largest is 1000 (which will be under-sampled to 100 for each epoch), the number of epochs needs to be increased by a factor of 10. This ensures that similar training dynamics will occur, and the model will have the opportunity to see all of the examples in the larger datasets.

In a further embodiment, which we will refer to as specialist and generalist training, the distillation step 230 is further modified. In this embodiment, and to further reduce costs, rather than training a student network serially on each teacher we propose to use each of the different teachers as students for every other teacher.

As previously described we form a single training cluster for training the Student model by establishing a plurality of inter-region peering connections between each of the nodes. As previously described the transfer dataset comprises each of the node datasets. However in this embodiment, the step of training the Student model 230 comprises training a plurality of Student models 234, wherein each Student model is trained by each teacher model at the node the teacher model was trained on. That is, each Teacher becomes a specialist in its own locality, and helps every other teacher to generalize to the data in every other locality. Once the plurality of Student models are trained (i.e., each student is trained under every other specialist), an ensemble model is generated from the plurality (or collection) of trained Student models.

In this embodiment, first, all Teachers are trained based on local data, i.e., Teacher Tᵢ becomes a specialist at location i (e.g., local clinic i) and trains to become optimized on the local data at this location. Then instead of having a Student sent to the location as in modified decentralized distillation, each Teacher is sent around sequentially to the other Teacher locations and learns/distils knowledge of other Teachers. Finally, all Teachers become generalists once they have been exposed to a sufficient number of locations (including all locations). Once this occurs they are considered trained models. In some embodiments, a threshold numbers of locations can be set for each Teacher to make it a generalist (or trained model). That is, it's a specialist, until it's been sent to a sufficient number of locations (i.e., exposed to more data) to turn it into a generalist. In some embodiments a Teacher is trained after it has been trained on a predetermined quantity of data at at least a threshold number of nodes. The final stage involves how to ensemble the weights of all trained Teachers together to make a final model.

Various ensemble methods may be used to generate the output AI model. In ensemble approaches a set of models are obtained, and then, each model ‘votes’ on input data (e.g., an image), and the voting strategy that leads to the best result is selected. These may include an Average Voting method, a weighted averaging method, a Mixture of Experts Layers (or learned weighting) method, or even using a further distillation method where a final model is distilled from the plurality of student models using simple distillation or modified decentralized distillation. This later method can be used to improve runtime inference efficiency (reducing costs) for a loss in accuracy (compared with the ensemble).

In decentralized training, the optimization of the model hyper-parameters occurs in two steps. If the distillation method is used, then each individual location is trained independently, including with different neural network architectures that are suitable for its own datasets (e.g., small models for smaller datasets, to prevent overfitting). The training of each Teacher model in its locality can be subject to its own hyper-parameter optimization suitable for its dataset. This optimization can be done individually. During the distillation process into a Student model, the hyper-parameters of the final Student model that is distilled from each Teacher model is also independent of the individual hyper-parameter optimizations of the Teacher models, and so can be treated as a normal hyper-parameter optimization problem as per usual neural network training. In the case of the decentralized training stage, where all model updates are sent to the master node, the hyper-parameter optimization problem must be treated collectively. The model copies on the slave nodes must be of the same architecture as the master model, and so a hyper-parameter optimization must occur at the master node level, which is sent to the slave nodes to update them. The hyper-parameter optimization can therefore be carried out as though the model was treated using normal distributed training, or even as though the data were placed on a single location, on a single large data set.

Table 4 present pseudocode for an embodiment of the Specialist and Generalist Distillation algorithm. Note that transfer dataset at each cluster central server, or at the global central server, is optional, as in practice it may not be possible to reserve a separate datasets to be used as a transfer set at each server. Thus these optional datasets are used if available to generate additional models which can be used to select the best cluster model (Mₖ) or best global model (M_{c}). Note that if no local transfer dataset Dₖ is available then step 29 is performed. Thus in step 30 one of Mₖ₁ and Mₖ₁ is available and the other is null. In one variation we modify step 29 so that Mₖ₂ is always calculated regardless of whether the transfer dataset is available. Then in step 30 we use Mₖ₂ or both Mₖ₁ and Mₖ₂ if available.

TABLE 4
Pseudocode for Specialist and Generalist Distillation Algorithm.

| | |
|---|---|
| 1 | Input: Data owner servers are divided into K clusters, Cₖ denotes k-th cluster |
| 2 | Input: Server Sᵢ is used for i-th data owner in each cluster |
| 3 | Input: Local dataset Dᵢ at Sᵢ, without external access |
| 4 | Input: Cluster Cₖ added with a cluster-based central server Sₖ, each with its own (optional) local |
| | (transfer) dataset Dₖ |
| 5 | Input: Global central server S_{c}, with its own (optional) local (transfer) dataset D_{c} |
| 6 | Input: Distillation loss function L |
| 7 | Input: R is number of times teacher model being sent around |
| 8 | |
| 9 | FOR k in range[K] |
| 10 | N ← sizeof(Cₖ) |
| 11 | FOR i in range[N] |
| 12 | Initialize AI teacher model n at Sᵢ |
| 13 | Train Mᵢ with the local data Dᵢ at Sᵢ until converged |
| 14 | ENDFOR |
| 15 | ENDFOR |
| 16 | FOR k in range[K] |
| 17 | N ← sizeof(Cₖ) |
| 18 | FOR i in range[N] |
| 19 | Locally make a copy of Mᵢ, denoted as Mᵢ' |
| 20 | FOR r in range[R], j in range[N] |
| 21 | IF compliance_check |
| 22 | Move Mᵢ' to Sⱼ and train Mᵢ' using distillation loss L |
| | on outcome of Mⱼ, and using local data Dⱼ |
| 23 | ENDFOR |
| 24 | IF compliance_check () |
| 25 | Move Mᵢ' to cluster central server Sₖ |
| 26 | ENDFOR |
| 27 | Initialize AI cluster-based model Mₖ at Sₖ |
| 28 | If local transfer data Dₖ exists then |
| | Mₖ₁ ← Train Mₖ using Distillation and loss L with all model Mᵢ' as teachers |
| | using its optional local transfer data Dₖ |
| 29 | else Mₖ₂ ← Send (Mₖ and all Mᵢ') to each node within cluster k, |
| | Mk is trained using distillation with each local node's data with teachers |
| | being all Mᵢ' |
| | // in one variation Mₖ₂ is always calculated (i.e., the “else” is deleted in step 29) |
| 30 | M_{kb} ← Best of [Mₖ₁, Mₖ₂] given one of optional transfer sets as the validation set |
| | // use whichever of Mₖ₁ or Mₖ₂ has been calculated or both if available (variation case) |
| 31 | IF compliance_check( ) |
| 32 | Move M_{kb} to global central server S_{c} |
| 33 | ENDFOR |
| 34 | Initialize a global model M_{c} at S_{c} |
| 35 | M_{c1} ← Train M_{c} using Distillation with distillation loss L and with all model M_{kb, k in K} as |
| | teachers and using its optional transfer data D_{c} |
| 36 | M_{c2} ← Send (M_{c} and M_{kb, k in K}) to each cluster k, M_{c2} is trained with each local transfer data |
| | using distillation with teachers being all M_{kb, k in K} (if there exists transfer datasets in any cluster |
| | central server) |
| 37 | M_{c3} ← Send (M_{c} and all Mᵢ' of cluster k) around nodes within cluster k, M_{c} is trained with each |
| | local node's data using distillation with teachers being all Mᵢ' ,M_{c} would travel by cluster |
| 38 | M_{cb} ← Best of [M_{c1}, M_{c2}, M_{c3}] given a set-aside validation set |
| | // this will be M_{c3} if there is no transfer dataset for the global central server or cluster central |
| | servers |
| 39 | RETURN M_{cb} |

The AI training process described herein can be summarized as each node trains a model, and then Knowledge Distillation is used to bring the individual models in different localities together into a single model. The various embodiments described herein give similar results but involve different time/cost trade-offs as is discussed further below. In some embodiments we may place some further conditions or constraints on the process.

In one embodiment, a first condition may be enforced that each node must contain more than a threshold number of data points (min data point threshold), or must be left out of the distillation process (i.e., Student model is not trained on that dataset). This threshold is dependent on the data set, and in one embodiment is obtained through training tests on the node using individual training.

Additionally, a second condition may be enforced such that if the number of nodes that do not contain the sufficient number of data points (as per the first condition) falls below a threshold number (min acceptable node threshold), then the proposed decentralized distillation step is ignored.

Decentralized training is then carried out on all the nodes. If distillation has been carried out (i.e., the second condition did not apply), then the distilled model is used as a pre-trained model prior to the Decentralized Training. This allows less total decentralized training to be carried out, since the distillation will have resulted in a model that is already close to a generalized and robust model, and the decentralized distillation process simply provides fine tuning of the model to improve the accuracy to the desired level as though the entire data set were combined on a single machine, but without ever removing the data from any node. This process reduces network traffic cost from transferring models as would be required from using Decentralized Training without first obtaining a single distillation model.

The process above can be summarized as: each node trains a model, and Knowledge Distillation can be used (if sufficient nodes contain sufficient data) to bring the individual models in different localities together into a single model, and then use it as a pre-trained model to carry out Decentralized Training.

However if Decentralized Training cannot be carried out due to scalability, that is, the total number of nodes is so large that logistically the process cannot be carried out, or it is undesirable for other reasons, then a multiple level process may be carried out as illustrated in FIG. 3 which is a schematic diagram of multi-level process 300 for performing decentralized training of an AI model according to an embodiment.

First, the N nodes 10 are separated into k clusters so that there is a finite and smaller number of clusters than the total number of nodes (k<N). Separating the nodes into a cluster may be performed deterministically, for example based on geographic proximity, via random selection, or a hybrid approach. For example the nodes may be partitioned into large geographic regions, with random allocation of a node with a geographic region to multiple clusters (within a region).

A decentralized training process 200, according to one of the above described embodiments is then performed separately in each of the k clusters (but not among or between the clusters) to generate a trained model for each cluster. That is we can define a cluster representative node that stores the cluster model that is the output of decentralized training within the cluster (and therefore representative of the cluster as a whole). The cluster representative node may be an administrative node for the cluster.

The resultant k models Mₛ₁, Mₛ₂, . . . , Mₛᵢ, . . . , Mₛₖ. (i.e., one for each cluster) can then be treated as separate nodes from this point on forming a first node layer 310 (i.e., the node layer is formed of the k cluster representative nodes, each storing the associated cluster model). A further decentralized training process 200 is carried out on this node layer 310 using only these k cluster representative nodes (i.e., the cluster models acts as a node). Effectively, the representative/administrative node of the cluster is treated as though it is a node with its own local dataset (which is under control of the representative/administrative node). From this point, only the k cluster representative (administrative) nodes participate in the training. They are treated for all intents and purposes using the same methodology as decentralized training as described above, except for just the specifically chosen k nodes. The administrative nodes contain information regarding the other nodes in its cluster prior to this step, as they are the resultant models from decentralized training exclusively within their respective cluster.

This multiple level process can be repeated multiple times, each time creating a new layer of clusters of nodes (over the previous nodes), and carrying out the decentralized training processing 200 as above, in this new layer. For example FIG. 3 shows a second layer 320 comprises of j models Mₛ₂₁, . . . , Mₛ₂ⱼ. These are then passed to a further decentralized training processing 200 to generate a final model Mₛ₃₁. This thus creates a hierarchical decentralized training system, in which each node is actually a cluster with an associated model trained on the underlying cluster until we are at lowest layer where the leaf nodes 10 correspond to actual nodes holding datasets.

One of the associated challenges with dealing with a decentralized environment is the provisioning of the cloud resources 51, 52, 54, 56, 58. To alleviate the issue of a cloud provisioning module 48 was developed for automatically provisioning the required hardware- and software-defined networking functionality. This was achieved by writing software that would search the available server configurations on a specific cloud services provider, and apportion the desired number of servers required, corresponding to the regions that have been selected (e.g., multiple regions in the United States on different sides of the continent, a node in Australia, etc.), at the same time. With the regions selected, the model configuration can be made available to each region through a distribution service, such as a secure git repository. This allows services to be created exactly as desired in any desired locality (e.g., cloud 50 hosting the administrative node 40, node clouds 61, 62, 64, 66), which automatically configure the training runtime and execute the training process. The server locations can be loaded to include only a local dataset, without sharing their dataset among other nodes. The training may then be synchronized among the batches, using distributed training functionality, now involving the separated servers. In the case of distillation, the training need not be synchronized, but the transfer set to be used collectively amongst the nodes can be apportioned. Inter-region peering connections can be established between nodes programmatically (i.e., using a software API) using node IDs and user IDs to establish connections and then setting appropriate routing rules (in a routing table) at each node using corresponding IP ranges (or routing IP addresses) for the connected nodes. For example AWS provide a powershell module and an API interface that provides commands for creating and accepting connection requests (e.g., createVpcPeeringConnection, acceptVpcPeeringConnection), to configure routing routes/tables for a connection (e.g., CreateRoute), and to modify or close down the connection (DeleteVpcPeeringConnection). Finally, after the training is complete, the provisioning module 48 can tear-down the configuration, that is, shut down all servers that were involved in this particular training run, and only these servers, thereby saving future costs on provisioned functionality (e.g., Hardware, Nodes, Networking features etc.).

In this document, the term Decentralized Training is used in the case where training of the model is performed on distributed data where data privacy is required to the extent such that:

(1) data is not moved or copied from its own locality, and must remain in its locality during training; and

(2) the trained AI model that is shared does not contain a copy or substantive copy of the data being trained on but only contains a general derivative of the data.

Embodiments of Decentralized Training methods described herein share some similarities with Federated Learning approaches, in that they still ensure data privacy, but have the advantage of not requiring the levels of encryption required for normal Federated Learning training. For example if Federated Learning was used to train an AI model on a device that contains private data and the model which is shared, it would be necessary in Federated Learning to encrypt the transmitted model weights, gradients, or data, in any combination, before the encrypted model potentially containing the private data is shared and aggregated at a different location/server with other encrypted models, also potentially containing private data, to create a generalized model. Note that in this case, although the private data is encrypted, it may still be shared and leave the host server in Federated Learning to create the AI model, which regulation or policies may not allow (and thus preventing use of Federated Learning). Accordingly in the Knowledge Distillation embodiments described herein, the model is trained (or informed) and aggregated at the source of the private data. Hence the model is less likely to comprise private data, and is also verified for data privacy before being shared thus improving over standard Federated Learning.

Results

This results section presents studies relating to the efficacy of the Decentralized Training technique, and the combined Knowledge Distillation Scaling Decentralized Training technique, described above.

The results are split into three parts: Performance Tests, which summarize how well the technique performs economically, in terms of time cost or monetary cost, Accuracy Tests, in which the ability of the technique to achieve the same accuracy benchmark as a similar model trained without decentralized training is measured, and Case Studies.

Finally, the potential time or money cost trade-off between Decentralized Training and Knowledge Distillation is discussed, with the optimal solution of the combined Distillation Scaling Decentralized Training approach being advocated.

In the first performance test, the normal deep learning training of residual network (ResNet) models implemented using the PyTorch deep learning library are customized to utilize the standard distributed multi-node multi-GPU training. This is the first step that would allow for training on datasets that are split across different server nodes. Note that off-the-shelf (OTS) distributed modules (e.g., the sub-module of PyTorch, torch.nn.distributed), which provide this base functionality, will only function within a single data center, e.g., US-Central-Virginia, or AUS-Sydney, and not between both at the same time, further highlighting the need for a fully decentralized methodology. The network cost for both distributed and decentralized training, however, is the same.

Performance Test 1 was conducted in the following steps:

P1-1: a fixed batch size of 16 is assigned for all tests;

P1-2: a fixed training, validation and test set is assigned for each test, with no randomization of the sets;

P1-3: a fixed server configuration is assigned, for benchmarking purposes. In this case, the AWS instance type G3.4×large is selected;

P1-4: the EC2 monitor web application available through AWS is used to sum the total network bytes transferred for each training run; and

P1-5: for each test, the following results are considered: 2 nodes in the same data center, 2 close-by nodes, 2 far-away nodes, 3 close-by nodes, 3 far-away nodes.

The results of server costs for these tests, excluding network traffic, are shown in Table 5. Note that ‘Batch time’ can be used as a proxy for network cost, as the network transfer will be much larger than the forward/backward passes of the network.

TABLE 5
A Summary of the Server Costs For Distributed Training, Which
Remains the Same For Decentralized Learning. Cost is in USD.

| | Data | Batch | Instance Cost | Network |
|---|---|---|---|---|
| Run | Partition | Time | (on demand) | Cost/batch |
| 1 N. Virginia | 100% | 0.251 s | $1.14/h | $0.004769 |
| 2 N. Virginia | 50/50 | 0.349 s | $1.14/h | $0.006631 |
| 3 N. Virginia | 40/30/30 | 0.543 s | $1.14/h | $0.01032 |
| Ohio, California | 50/50 | 2.501 s | $1.14/h, $1.534/h | $0.05573 |
| (east to west) | | | | |
| California, Oregon | 50/50 | 0.928 s | $1.534/h, $1.14/h | $0.02068 |
| (2 in west) | | | | |
| Ohio, California, | 35/35/30 | 3.038 s | $1.14/h, $1.534/h, | $0.06470 |
| Oregon | | | $1.14/h | |

The most important outcome from Table 5 is that as the number of nodes and especially cross-region nodes increases, the time taken to compute a batch, increases drastically. This length of time taken to reach a specific epoch number therefore increases the total length of time servers must be running. This is the source of the high cost of distributed or decentralized training used alone, without distillation.

Now the cost of the network traffic per batch can also be summarized. This is dependent on the neural network architecture, which determines the size of the file to be transferred.

In the second performance test, an example of training a ResNet-50 neural network to epochs is considered.

Performance Test 2 was conducted in the following manner, noting the final cost:

P2-1: a single training epoch contains 436 batches, and 16 images in one batch;

P2-2: the total traffic from the instance server is: 41.6 GB inbound and 41.6 GB outbound;

P2-3: for a single batch (regardless of its size), the network sends in and out 41.6 GB/436=97.7 MB (which is approximate the whole ResNet-50 as a saved file);

P2-4: the cost per batch for transferring data between 2 nodes in the same availability zone is free;

P2-5: the cost per batch for transferring data between nodes in 2 availability zones in 1 region is 0.0977*0.01*2=$0.002; and

P2-6: the cost per batch for transferring data between 2 inter-region nodes: 0.0977*0.02*2=$0.004.

Therefore, for 2 nodes inter-region, the cost should be as follows: 41.6*10*$0.02*2 (1 worker+1 master)=$16.64. For 3 nodes inter-region, the cost should be as follows: 41.6*10*$0.02*4 (2 workers+1 master)=$33.28. Adding 1 more worker node will add $16.64 to the cost.

Furthermore, the cost will increase when using other commonly used but larger neural networks like DenseNet-121 or ResNet-152, in proportion to the file size of the models. In many cases, this can be double price as listed above. Furthermore, this example covers a base case of only about 4.5k iterations. Thorough model training can take up to 1M iterations.

This is a scaling issue, that is, it can be seen that the total cost of distributed or decentralized training, used alone, can increase rapidly, becoming uneconomical if the training becomes large scale, which is likely to occur for large scale projects across industries. Therefore, the cost can be made economical and manageable using Knowledge Distillation for Scaling Decentralized Training together.

By making use of Knowledge Distillation, there is a cost trade-off between normal decentralized and distillation, since the distillation can be achieved without the network traffic and server costs associated with batch-by-batch reporting, as described above. An estimate of the cost saved by this combined method is roughly two orders of magnitude, but is dependent on the size of the model, the number of updates, and the distance between the nodes. A worked example is described below.

Consider a scenario where distillation training requires 60 weight transfer operations. For a single decentralized training run, an epoch would contain 5216/16=326 and 9013/16=563 batches of size 16 (for the above setting). The number of data/weight transfer back and forward for a single batch is 4*2=8, given 4 workers and a master node. For a model trained with 200 epochs, the total numbers of weight transfers is 326*8*200=521,600 and 563*8*200=900,800 times, for two example data sets of chest X-ray and skin cancer datasets. Hence, by using the distillation training only, while the accuracy is only dropped less than 1%, the average number of transfers saved are 521,600/60=8,693 and 900,800/60=15,013 times for two datasets, respectively. Refer to Table 5 to obtain the total cost per transfer.

Accuracy tests were also performed. In the first accuracy test, the training of a neural network is repeated for a number of scenarios, each testing the additions to the decentralized training process to improve accuracy as outlined in the Proposed Solution section above.

Accuracy Test 1 was conducted with no loss function weighting being used, in order to test the class distribution. It was conducted to consider the following scenarios, for 2 nodes:

A1-1: the data is balanced among nodes, and the class distribution is balanced within each node;

A1-2: the data is unbalanced among nodes, and the class distribution is balanced within each node;

A1-3: the data is balanced among nodes, and the class distribution is unbalanced within each node; and

A1-4: the data is unbalanced among nodes, and the class distribution is unbalanced within each node.

Accuracy Test 2 is a repeat of Accuracy Test 1, but with 3 nodes instead of 2. These scenarios are labelled A2-1, A2-2, A2-3 and A2-4 to correspond to the above scenarios, respectively.

For each test, the best accuracy achieved is quoted.

Using an example of an embryo viability assessment model, a summary of the change in accuracy compared to a baseline is shown in Table 6, in which the model is trained on a single server.

TABLE 6
A Summary of Accuracies For Training an Embryo Viability
Assessment Model, Compared to a Pre-Defined Baseline Accuracy.
A Pre-Calculated Baseline Test Accuracy of 62.14% is Used.

| | | Difference from |
|---|---|---|
| Test | Test Accuracy (negative/positive) | Baseline |
| Baseline | 62.14% (46.76%/77.30%) | |
| A1-1 | 62.86% (56.12%/69.50%) | +0.72% |
| A1-2 | 62.50% (54.68%/70.21%) | +0.36% |
| A1-3 | 62.14% (57.55%/66.67%) | 0.00% |
| A1-4 | 58.57% (81.29%/36.17%) | −3.57% |
| A2-1 | 62.86% (66.19%/59.57%) | +0.72% |
| A2-2 | 62.14% (61.87%/62.41%) | 0.00% |
| A2-3 | 62.50% (52.52%/72.34%) | +0.36% |
| A2-4 | 58.21% (80.58%/36.17%) | −3.93% |

By carrying out this study, the number A1-3, A1-4, and A2-3, A2-4 are the worst case scenario, with A1-4 and A2-4 being by far the worst, which matches the most likely real-world situation, where each clinic has different amounts of data in which the viable/non-viable images are significantly unbalanced.

Overall Results for Different Scenarios of Unbalancing are as follows:

A1-1 & A2-1 accuracy: ranked Best (may be better than single node—traditional training);

A1-2 & A2-2 accuracy: ranked Good;

A1-3 & A2-3 accuracy: ranked Good (training on each node may bias on viable or non-viable class samples, the final model performs fine, IF SUM(viable images) SUM(non-viable images); and

A1-4 & A2-4 accuracy: ranked Poor.

Accuracy Test 3 compares a range of scenarios in which multiple weighting methods are combined together, and the optimal number and types of loss weightings are obtained. The three scenarios are:

A3-1: Sample/image level weighting: we put more weight on the samples that were hard to classify and decrease the impact on easy correct predictions. Mathematically, a scaling factor is added to the cross-entropy loss function;

A3-2: Class level weighting: in the cases of unbalanced class distribution, we want to put more weight on the class that has a smaller number of samples; and

A3-3: Distributed node level weighting: in the decentralized training, the model and data are copied and separated between a number of nodes/computers, possibly inter-region. The amount of data available on each node can be significantly unbalanced.

The overall results for different 3-level weighting loss scenarios was as follows.

When training on the unbalanced node data, unbalanced class distribution, the class weighting and node weighting level individually help to boost the accuracy to 1-2%.

When training on the balanced class distribution, the sample level weighting helps to boost the accuracy to about 1%.

In terms of combining different weighting levels, the sample/image level and node level weighting, used together, would help for the current experimental configuration. All other combinations do not provide a benefit.

By making use of Knowledge Distillation, there is an accuracy trade-off between normal decentralized and distillation, since the distillation generally will not achieve 100% of the accuracy results achievable through normal training, distributed training or decentralized training. An estimate of the achievable accuracy of the distillation training method is a drop of 1.5% on average compared with the distributed training or decentralized training results. The proposed modified decentralized distillation or specialist and generalist method can help to produce similar prediction accuracy to the normal distributed training or decentralized training.

We now describe some additional case studies using embodiments of the decentralized training methodology as described herein. In the first case a simple ‘cat or dog’ classification problem is performed, involving both ‘clean’ and ‘noisy’ labels. This introduction of noise into the training, validation and test datasets ensures that the model does not reach maximum accuracy automatically, and thereby demonstrates the difference in the decentralized and centralized training approaches, both in accuracy (total number of examples correct), and their ability to handle and to some extend overcome differing levels of noise.

The experiment is tested in a number of settings: a simple 5-node scenario, in a single cluster where data is equally spread among the nodes. The methodology for introducing noise into the data, and practical comparisons as to the choice of (optional) transfer sets is explored. The experiments are then extended to a 15-node scenario, in a single cluster, showing consistent results. This assists in selection practical bounds on the total number of (node-level) ‘epochs’ to consider when finalizing the training process across multiple nodes using distillation training, and also reasonable parameters to select for the ‘alpha’ parameter between teacher and student (specialist and generalist) models in this setting. The 15-node scenario is then divided into three equal clusters using the clustering method described above demonstrating an offset in the performance increases seen from the experiments above. The trade-off between data transfer (network) cost, and model accuracy is also further explored, thus providing a guide as to how to optimize decentralized training for real world experiments.

The dataset used for the following experiments was a set containing images of dogs and cats, taken from ImageNet. 4500 images were used for training and validation set, 4501 images were used as the test set. The original data is considered clean data. The noisy data set was created by converting 10% of dog images to the “cat” class label (class 0), and 50% of cat images to the “dog” label (class 1). This would result in the amount of noise appeared in cat and dog classes, respectively being 17% and 36%. Therefore, we now have a clean baseline training-validation set and a noisy baseline training-validation set. The test set is kept as clean as the original images. The detailed number of images available in each class is listed in Table 7.

TABLE 7
Clean and Noisy Train-Validation-Test Cat = Class 0, Dog = Class 1

| | Data | | | | | |
|---|---|---|---|---|---|---|
| | Clean train-val | | Noisy train-val | | Fixed clean test se | |
| Classes | class 0 | class 1 | class 0 | class 1 | class 0 | class 1 |
| # Images | 2250 | 2250 | 1350 | 3150 | 2253 | 2248 |
| | | | (added 17% noise) | (36% noise) | | |

| | | | |
|---|---|---|---|
| Total | 4500 | 4500 | 4501 |

The following experiments require the existing data at each node in the decentralized training regime, as described above and outlined in Table 4. We choose 5 nodes and 15 nodes based decentralized training experiments. In the case of 5 nodes, each node would contain 900 images, with either clean or noisy data. The approach to create noisy data for the 5 nodes situation is exactly the same as the creation of the noisy baseline dataset. In the case of 15 nodes, only the noisy data is created for each node. 300 images are available at each node with only 90 images labelled as cat and 210 images labelled as dog. For any number of nodes, the number of images summed up from all node's data is equivalent with the size of the baseline dataset. This would make a fair comparison between the baseline model and the decentralized model, training on baseline datasets and the nodes' data, respectively. More details about the dataset summary can be seen in Table 8.

TABLE 8
Data Allocated at Each Node

| | Data | | | | | |
|---|---|---|---|---|---|---|
| | Data of 1 node in 5 (clean) | | Data of 1 node in 5 (noisy) | | Data of 1 node in 15 (noisy) | |
| Classes | class 0 | class 1 | class 0 | class 1 | class 0 | class 1 |
| # Images | 450 | 450 | 270 | 630 | 90 | 210 |
| | | | (with 17% noise) | (with 36% noise) | (with 17% noise) | (with 36% noise) |

| | | | |
|---|---|---|---|
| Total | 900 | 900 | 300 |

The model architecture used for the following experiments is resnet18, with the pre-trained model using the ImageNet dataset. Since the ImageNet data contain the dog and cat images/classes, a training model is expected to perform quite well at the very first training epochs. After several epochs, the model would become more biased toward the provided training set and then give sensible results on the validation and testing sets. Hence, in selecting the best model for comparison, the model is more appropriately selected after at least 15-30 epochs of training.

The network parameters are selected by running multiple runs using the baseline clean dataset. The best set of parameters such as learning rate, regularization methods, weight decay, loss function, or batch size, etc., was identified and then used throughout all the experiments for decentralized training.

The model in this particular scenario is selected using the best balanced accuracy on the validation set (this is a ‘set aside’ set from the training set—normally it accounts for 20% of the training set). All the results reported here are for the testing set. The term “Best on validation set” means that the testing results are shown based on the best balanced accuracy chosen on the validation data. On the other hand, the term “Best on test set” means that the results presented are the best balanced accuracy selected on the test set only.

The following tables present testing experimental results with 5 evaluation metrics, including mean accuracy, class 0 accuracy, class 1 accuracy, balanced accuracy and log loss.

Case Study 1: 5 Nodes Single Cluster

The baseline models were trained for at least 100 epochs and the best model based on the validation set results would be selected for comparison.

In this experiment, there are 5 nodes and single cluster. The training procedure includes following steps: (1) At each node, a teacher/specialist model is trained with local data for epochs; (2) A student/generalist model is created at each node (after all the teachers being trained), for simplicity the student model is a copy of the local teacher model; (3) The student model is sent around to other nodes, and at each node it learns the local data and distils knowledge from the local teacher; (4) A final model is created and a copy of all trained students is made available at each node. The final model A will travel to each node and learn the local data via the distillation approach using the ensemble of all trained students. If there exists the transfer dataset for those 5 nodes, another final model B is trained using the transfer dataset via distillation approach using the ensemble of all trained students. The final decentralized model will be the best of model A and B.

Basic Model Training

In the following, (4) will be tried with different approaches to the choices of transfer dataset. The point is, in practice, creating a reasonable sized transfer set would be more difficult, as it needs to be contained within a separate server since the data cannot be moved. There are two approaches here:

Dc-1: we create a new final model and that learns to distil knowledge from all trained students using a separate transfer set (which is different from any training, validation and testing set, the transfer set is clean and contains 2000 images)

Dc-2: we create an ensemble model using 5 trained students at the end

These results confirm the decentralized training algorithm works quite well with just a minimal difference in accuracy between decentralized training approaches and the baseline using the clean datasets. Firstly we found that using the transfer set (in Dc-1) can perform better than the ensemble approach (Dc-2) in terms of using the noisy datasets. Secondly and importantly, we found that the decentralized approach (Dc-1) can actually outperform the baseline, which is the centralized AI training approach. The experiment was repeated multiple times using different dataset configurations, and the same improved accuracy result was achieved using decentralized training (additional results relating to this are discussed in the next section).

This result was unexpected and significant in demonstrating the utility of our decentralized training approach for both data privacy, performance (accuracy and generalizability), and ability to robustly train in the presence of noisy data which is likely to occur in a decentralized situation where there are multiple data owners and servers and lack of global data transparency.

All the local specialist model at each node results in less accurate in model generalization compared with the baseline model since the local specialist models have access to much smaller sets of training data than the baseline training set.

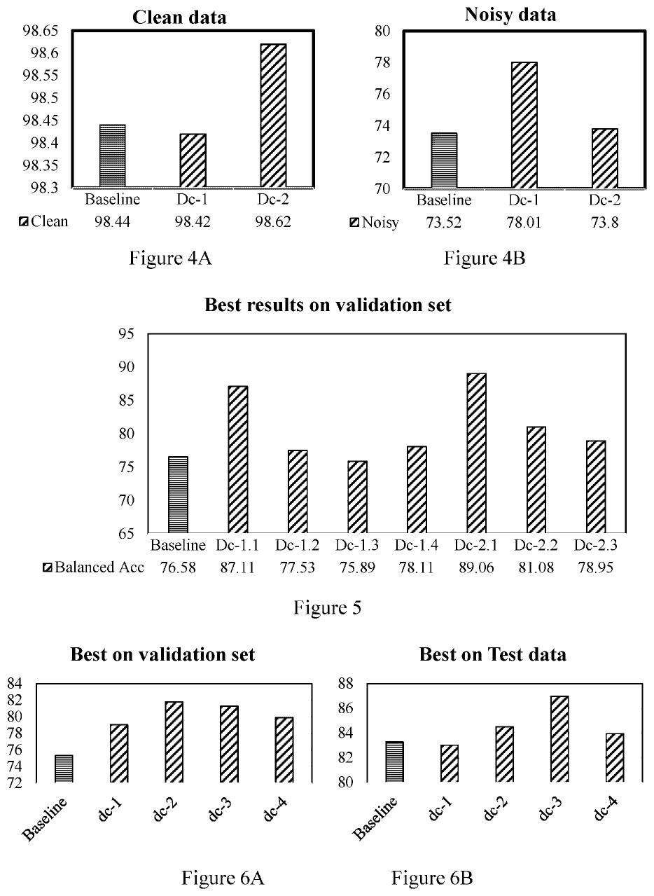

Table 9 shows all the mentioned models' results while FIGS. 4A and 4B presents the balanced accuracy result comparison for clean data (FIG. 4A) and noisy data (FIG. 4B) as balanced accuracy is selected as the key metric to assess the model's performance.

TABLE 9
Model Result Comparison: Baseline and Others.

| | Mean | | | Balanced | |
|---|---|---|---|---|---|
| Metrics | Acc | Class 0 | Class 1 | Acc | Log loss |
| Clean data | | | | | |
| Baseline | 98.44 | 98.62 | 98.26 | 98.44 | 0.061 |
| Dc-1 | 98.42 | 98.49 | 98.35 | 98.42 | 0.0455 |
| Dc-2 | 98.62 | 98.13 | 99.11 | 98.62 | 0.0385 |
| Noisy data | | | | | |
| Baseline | 75.31 | 57.34 | 93.32 | 75.33 | 1.2356 |
| Dc-1 | 78 | 57.68 | 98.35 | 78.01 | 0.4211 |
| Dc-2 | 73.78 | 48.49 | 99.11 | 73.8 | 0.598 |
| Noisy data | | | | | |
| Model on node 1 | 70.56 | 45.31 | 95.86 | 70.59 | 1.2733 |
| Model on node 2 | 68.42 | 40.3 | 96.61 | 68.46 | 1.4826 |
| Model on node 3 | 73.05 | 48.42 | 97.73 | 73.07 | 1.131 |
| Model on node 4 | 73.02 | 48.91 | 97.19 | 73.05 | 1.1544 |
| Model on node 5 | 69.25 | 41.54 | 97.01 | 69.28 | 1.5061 |

As can be seen in Table 9 the experimental results for the clean training set reach a ceiling and there remains no further interesting results. Thus in the following experiments all the results are associated to the noisy training-validation datasets.

We take a further investigation on the options available for the choice of transfer set by performing additional decentralized training experiments on different “transfer sets,” In practice, a transfer set might not be available, thus the existing data at each node can play a role as the transfer set. Table 10 and 11 present the results of a simple test to explore what happens when the transfer set is only a single node's data (Dc-1.2,1.3,1.4), a combination of all the nodes' data (Dc-1.1) or all the separate nodes' data (multiple transfer set cases in Dc-2.x). Using the combination of all the nodes' data as the transfer set is not typically available as an option in reality, since the data cannot leave the owner/node server due to security or privacy reasons, hence this approach is intended to mimic the real world situation, and to answer the question of whether there any benefits when using all nodes' data together as the transfer set. This is a pre-cautious step before we conduct a costly model traveling around the world and in turn taking each node's data as a transfer set (Dc-2.x in Table 10 and 11). At each node, it is required that a copy of all the trained student models is made available. The final model is trained on the local data (seen as a transfer set) and consulting the knowledge from the plurality of trained students (now they become teachers for the final model). The process may require extensive data transfer globally and server spin-up on cloud services. The following experiments would focus on reducing the travelling and training cost by sending the final model and all trained students around the world once only.

Table 10 compares the results of all above decentralized settings with different transfer sets. The results for training Dc-1.1 with combined all nodes' data as the transfer set shows a significant 9-11% improvement in accuracy compared with other experiments Dc-1.2, 1.3 and 1.4 which use a single separate/node data as transfer set (see FIG. 5). Again, the performance of all the decentralized AI training experiments outperformed the baseline result. The interesting point here is that even if the transfer set is as small as a single node's data, the Dc-1.2, 1.3 and 1.4 results are more or less similar with the baseline result.

In terms of using multiple transfer sets (all nodes' data together, or one-by-one in a decentralized manner), the three settings consider the variation of the number of training epochs of the final model at each node from 5 to 20. The testing balanced accuracy decreases when the final model remains longer at each node since the final model is prone to overfitting and tends to ‘forget’ what it learned in the past. Especially, training 5 epochs at each node (Dc-2.1) gives the best generation performance which is about 11% better than the baseline results (see FIG. 5).

TABLE 10
Comparison of Decentralized Model Results for Different Transfer Set Scenarios.

| | | | | | | |
|---|---|---|---|---|---|---|
| Exp-id | Description | Mean Acc | Class 0 | Class 1 | Balanced Acc | Log loss |

| | | | | | |
|---|---|---|---|---|---|
| Baseline | 75.31 | 57.34 | 93.32 | 75.33 | 1.2356 |

Using a single separate transfer set

| | | | | | | |
|---|---|---|---|---|---|---|
| Dc-1.1 | Combining all nodes' data | 87.11 | 85.35 | 88.87 | 87.11 | 0.3031 |
| | as a transfer set | | | | | |
| Dc-1.2 | Node 1 data as transfer set | 77.51 | 58.18 | 96.88 | 77.53 | 0.4933 |
| Dc-1.3 | Node 2 data as transfer set | 75.87 | 53.83 | 97.95 | 75.89 | 0.5666 |
| Dc-1.4 | Node 3 data as transfer set | 78.09 | 58.18 | 98.04 | 78.11 | 0.4926 |

Using multiple transfer sets (including local data at each node)

| | | | | | | |
|---|---|---|---|---|---|---|
| Dc-2.1 | 5 Epochs at each node | 89.06 | 92.94 | 85.18 | 89.06 | 0.3379 |
| | (batch size 8) | | | | | |
| Dc-2.2 | 10 epochs at each node | 81.07 | 71.81 | 90.34 | 81.08 | 0.415 |
| | (batch size 16) | | | | | |
| Dc-2.3 | 20 epochs at each node | 78.93 | 65.15 | 92.74 | 78.95 | 0.4367 |
| | (batch size 32) | | | | | |

As can be seen in Table 9 the experimental results for the clean training set reach a ceiling and there remains no further interesting results. Thus in the following experiments all the results are associated to the noisy training-validation datasets.

Case Study 2: 15 Nodes Single Cluster

To improve the scalability of the decentralized AI technique, we use clusters of nodes. The decentralized AI training runs on each individual cluster of nodes, and then runs between clusters as a single node (i.e., in a hierarchical way), to reduce the overall number of nodes that need to run the decentralized AI training. To test the efficacy of the hierarchical clustering approach, we ran experiments to test the differences between running all 15 nodes as a single cluster and running 15 nodes as three separate clusters.

We consider two scenarios including the single cluster setting and 3 cluster setting (5 nodes in each cluster). The results of these two scenarios would give us an indication to how much the clustering of nodes impacts the final model's generalization capability and performance.

In this case study, a single cluster is considered. What is different here compared with the 5-node experiments (case study 1) is:

The dataset available at each node is much smaller (300 images)

The teacher/specialist model at each node is only exposed to 300 images which is deliberately chosen to be on the lower limit to assure a good teacher model

When training the final model, all 15 trained students are used for the distillation strategy, hence the training process would take longer and more memory is required to load all these 15 trained models

The final model should remain at each node shorter time (fewer number of epochs, compared with the 5-node case) because there is not much data to learn.

Case Study 2.1 Standard 15 Nodes with a Single Cluster

Table 11 shows the results of the final model after it was sent around to each node once. The number of epochs at each node was ranged from 3 to 10. If the final model remains at each node 3 epochs, the total number of training epochs in its lifetime is 3*15=45 epochs which is below half the number of epochs being trained for the baseline model. Table 11 and FIGS. 6A and 6B which is a comparison of the baseline result to different decentralized experiments, give the testing result associated with the best balanced accuracy on the validation set (FIG. 6A), and the best testing result itself to see the model's predictive capability on the test set (FIG. 6B). In both cases, the decentralized models outperformed the baseline results, especially when the final model is trained 5-8 epochs at each node, which accounts for around 5% improvement in accuracy.

These results re-assure that in the single cluster case, either with 5 nodes or 15 nodes, the decentralized model is superior over the traditional single server (centralized) baseline training.

TABLE 11
Comparison of Decentralized Model Results For Different Number of Epoch Scenarios.

| | | | | | | |
|---|---|---|---|---|---|---|
| Exp-id | Description | Mean Acc | Class 0 | Class 1 | Balanced Acc | Log loss |

Best on validation set

| | | | | | | |
|---|---|---|---|---|---|---|
| Baseline | | 75.31 | 57.34 | 93.32 | 75.33 | 1.2356 |
| Dc-1 | 3 epochs at each node | 79.04 | 70.83 | 87.27 | 79.05 | 0.4726 |
| | (3*15 = 45 epochs) | | | | | |
| Dc-2 | 5 epochs at each node | 81.78 | 68.22 | 95.37 | 81.79 | 0.3971 |
| | (75 epochs) | | | | | |
| Dc-3 | 8 epochs at each node | 81.29 | 80.15 | 82.42 | 81.29 | 0.4492 |
| | (90 epochs) | | | | | |
| Dc-4 | 10 epochs at each node | 79.89 | 66.4 | 93.41 | 79.9 | 0.4356 |
| | (150 epochs) | | | | | |

Best on test

| | | | | | | |
|---|---|---|---|---|---|---|
| Baseline | | 83.27 | 71.06 | 95.5 | 83.28 | 0.9057 |
| Dc-1 | 3 epochs at each node | 83.02 | 79.58 | 86.47 | 83.02 | 0.429 |
| | (3*15 = 45 epochs) | | | | | |
| Dc-2 | 5 epochs at each node | 84.51 | 79.98 | 89.05 | 84.51 | 0.4092 |
| | (75 epochs) | | | | | |
| Dc-3 | 8 epochs at each node | 86.98 | 86.1 | 87.85 | 86.98 | 0.3757 |
| | (90 epochs) | | | | | |
| Dc-4 | 10 epochs at each node | 83.93 | 73.72 | 94.17 | 83.94 | 0.3779 |
| | (150 epochs) | | | | | |

Influence of Teacher Models on the Student Prediction Accuracy

In this section, we will investigate the influence of teachers' model on the student prediction accuracy. When the final model travels to each node (or the representative node of each cluster), it learns the local data based on the ensemble knowledge of multiple teachers. Since the distillation loss function composes the teachers' output component and the output component of the final model itself, the (representative) ‘alpha’ parameter value in the loss function controls the level of teachers' effect on the loss outcome. The larger the alpha value, the more influence the final model would receive from teachers. Alpha=0 means that there are no teachers used. Alpha=1 means that student model relies completely on the teachers' outcome. In practice, teachers assist with training, except when teachers were trained with low-quality/inefficient local training set (model being prone to over-fitting or untrainable). In that case, the best option is to reduce the influence of teachers' output on the student model training to an appropriate level.

In Table 12, the alpha values range from 0 to 0.7, several decentralized training runs were conducted, and the averaged results associated with each alpha value are reported. It can be seen that the alpha=0.3 is preferred for this experiment. Using teachers for student distillation training is beneficial if we know how to choose the right distillation loss function's parameters.

TABLE 12
Teacher Ensemble Influence on Student Model's Performance

| | | | | | | |
|---|---|---|---|---|---|---|
| Exp-id | Description | Alpha = 0.0 | Alpha = 0.1 | Alpha = 0.3 | Alpha = 0.5 | Alpha = 0.7 |

Best on validation set

| | | | | | | |
|---|---|---|---|---|---|---|
| Dc-1 | 3 epochs at each node | 78.88 | 73.46 | 79.05 | 76.52 | 74.53 |
| Dc-2 | 5 epochs at each node | 75.42 | 79.03 | 74.38 | 80.22 | 69.51 |
| Dc-3 | 8 epochs at each node | 72.69 | 73.44 | 81.29 | 79.86 | 68.4 |
| Dc-4 | 10 epochs at each node | 80.7 | 79.11 | 79.91 | 80.6 | 78.57 |
| AVERAGE | | 76.9225 | 76.26 | 78.6575 | 79.3 | 72.7525 |

Best on test

| | | | | | | |
|---|---|---|---|---|---|---|
| Dc-1 | 3 epochs at each node | 78.88 | 77.21 | 83.02 | 85.16 | 82.13 |
| Dc-2 | 5 epochs at each node | 84.62 | 83.51 | 84.51 | 86.6 | 85.37 |
| Dc-3 | 8 epochs at each node | 83.23 | 80.49 | 86.98 | 79.86 | 84.82 |
| Dc-4 | 10 epochs at each node | 85.56 | 81.37 | 85.41 | 80.6 | 86.73 |
| AVERAGE | | 83.0725 | 80.645 | 84.98 | 83.055 | 84.7625 |

Case Study 3: 15 Nodes Divided into 3 Clusters

In this case study, 3-cluster setting will be used. What is different here compared with the 15-node and single cluster experiments:

The cluster specific generalists: when a teacher is available at each node, a new student/generalist will travel around its cluster container. The student becomes a generalist for that specific cluster. There are 5 trained students/generalists for each cluster here, as we have chosen to equally divide the nodes among the clusters for this experiment.

At the final stage, the final model jumps from cluster to another cluster until all clusters have been visited. At each cluster, it is trained exactly same as in the 5-node situation as in Case study 1 (travels to each node, learns the node local data, distils knowledge of all container cluster's trained students/generalists. This is required to copy all the trained students/generalists within mentioned cluster to each node).

Standard 15-Node with 3-Cluster Experiments

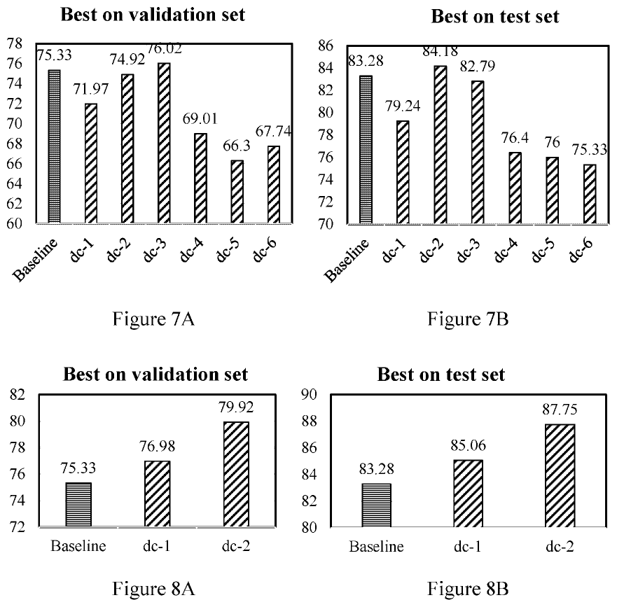

Table 13 lists all the experimental results when training several decentralized models. The number of epochs for the final model remaining at each node ranges from 2 to 20. The final model accuracy dropped by approximately 2-3% and sometime more (if the final model remains longer at each node, say the number of epochs is larger than 8) compared with the baseline result (see FIG. 7 which is a comparison of baseline result and different decentralized experiments). We found that the clustering of nodes resulted in a drop in accuracy as anticipated. The main reason is that generalist model has access only to data available in the given cluster. Hence only the final model has access to all data across each cluster. However, it seems that a single visit to each cluster would not be enough for the final model to have comparable performance with the baseline.

The size of node's data is important, but these results indicate that clustering is the main factor contributing to the drop in the final model's accuracy.

TABLE 13
Decentralized Models Comparison.

| | | | | | | |
|---|---|---|---|---|---|---|
| Exp-id | Description | Mean Acc | Class 0 (Cat) | Class 1 (Dog) | Balanced Acc | Log loss |

Best on validation set

| | | | | | | |
|---|---|---|---|---|---|---|
| Baseline | | 75.31 | 57.34 | 93.32 | 75.33 | 1.2356 |
| Dc-1 | 2 epochs at each node | 71.98 | 83.22 | 60.72 | 71.97 | 0.5792 |
| | (2*15 = 30 epochs) | | | | | |
| Dc-2 | 5 epochs at each node | 74.91 | 66.79 | 83.05 | 74.92 | 0.5033 |
| | (75 epochs) | | | | | |
| Dc-3 | 8 epochs at each node | 76.01 | 58.01 | 94.03 | 76.02 | 0.4906 |
| | (90 epochs) | | | | | |
| Dc-4 | 10 epochs at each node | 68.98 | 45.13 | 92.88 | 69.01 | 0.6607 |
| | (150 epochs) | | | | | |
| Dc-5 | 15 epochs at each node | 66.27 | 40.65 | 91.94 | 66.3 | 1.0482 |
| | (300 epochs) | | | | | |
| Dc-6 | 20 epochs at each node | 67.71 | 47.18 | 88.3 | 67.74 | 0.8264 |
| | (300 epochs) | | | | | |

Best on test set

| | | | | | | |
|---|---|---|---|---|---|---|
| Baseline | | 83.27 | 71.06 | 95.5 | 83.28 | 0.9057 |
| Dc-1 | 2 epochs at each node | 79.22 | 66.66 | 91.81 | 79.24 | 0.4848 |
| | (2*15 = 30 epochs) | | | | | |
| Dc-2 | 5 epochs at each node | 84.18 | 84.15 | 84.2 | 84.18 | 0.4391 |
| | (75 epochs) | | | | | |
| Dc-3 | 8 epochs at each node | 82.78 | 74.07 | 91.5 | 82.79 | 0.3959 |
| | (90 epochs) | | | | | |
| Dc-4 | 10 epochs at each node | 76.38 | 60.8 | 91.99 | 76.4 | 0.5058 |
| | (150 epochs) | | | | | |
| Dc-5 | 15 epochs at each node | 76 | 68.84 | 83.18 | 76 | 0.5445 |
| | (300 epochs) | | | | | |
| Dc-6 | 20 epochs at each node | 75.31 | 60.49 | 90.16 | 75.33 | 0.594 |
| | (300 epochs) | | | | | |

Trade-Off Between Data Transfer and Model Accuracy

There is a certain concern that when the final model is sent from one node to another, the network transfer and server costs may increase significantly. Since the clustering is inevitable in the real-world situation, the larger the number of clusters may further reduce the final model's prediction accuracy. The following experiments will confirm that when the final model is allowed to travel to each node or cluster more than once, the model generalization is able to increase to an acceptable level (say to be comparable with the baseline). We found that when the final decentralized model visits each node more than three times, the final prediction accuracy boosts to be better than the baseline results by more than 4% in balanced accuracy (see Table 14 and FIGS. 8A and 8B). If we allow the final model to visit each node five times, we can set the number of node-level epochs (the time to remain at each node) small enough such as 1 or 2. In this case the 2 epochs option is preferred.

TABLE 14
Comparison of Decentralized Models For Different Numbers of Epochs.

| | | | | | | |
|---|---|---|---|---|---|---|
| Exp-id | Description | Mean Acc | Class 0 (Cat) | Class 1 (Dog) | Balanced Acc | Log loss |

Best on validation set

| | | | | | | |
|---|---|---|---|---|---|---|
| Baseline | | 75.31 | 57.34 | 93.32 | 75.33 | 1.2356 |
| Dc-1 | 1 epoch at each node | 76.98 | 75.01 | 78.95 | 76.98 | 0.5758 |
| | (1*15*5 = 75 epochs) | | | | | |
| Dc-2 | 2 epochs at each node | 79.91 | 71.01 | 88.83 | 79.92 | 0.5204 |
| | (150 epochs) | | | | | |

Best on test

| | | | | | | |
|---|---|---|---|---|---|---|
| Baseline | | 83.27 | 71.06 | 95.5 | 83.28 | 0.9057 |
| Dc-1 | 1 epoch at each node | 85.04 | 73.5 | 96.61 | 85.06 | 0.4076 |
| | (75 epochs) | | | | | |
| Dc-2 | 2 epochs at each node | 87.75 | 90.14 | 85.36 | 87.75 | 0.4162 |
| | (150 epochs) | | | | | |

There thus exists a trade-off between network cost in transferring the model to different nodes a sufficient number of times, and how much (acceptable) percentage does the final prediction model drop below the baseline model's accuracy. It is clear that the final model exhibits higher performance when it has greater chance and enough time to learn from data at each node.

Once models are trained they may then be deployed on computational systems to analyze or classify new images or datasets. In some embodiments deployment comprises saving or exporting the trained AI model, such as by writing the model weights and associated model metadata to a file which is transferred to the operational computation system and uploaded to recreate the trained model. Deployment may also comprise moving, copying, or replicating the trained model onto an operational computational system, such as one or more cloud based servers, or locally based computer servers at local sites (e.g., healthcare clinics). In one embodiment deployment may comprise reconfiguring the computational system the AI model was trained on to accept new images or data and generate a diagnosis or condition using the trained model, for example by adding an interface to receive medical data (e.g., image or diagnostic datasets), execute/run the trained model on the received data, and to send the results back to the source, or to store the results for later retrieval. The deployed system may be a cloud based computational system or a local computational system. A user interface may be provided to allow a user to upload data to the computational system (and trained AI model) and then received results from the model (e.g., report or datafile) which can then be used, for example to make a medical or clinical decision.

The AI model may be deployed in distributed systems with separate computer systems at the user and administrator (central node) sides. Thus in one embodiment there is provided a cloud based computation system for generating an AI based assessment from one or more images or datasets. The cloud based computation system may comprise one or more computational servers comprising one or more processors and one or more memories configured to store an Artificial Intelligence (AI) model configured to generate an assessment from one or more images or datasets wherein the AI model is generated according to an embodiment of the methods as described herein. The one or more computational servers are configured to:

receive, from a user via a user interface of the computational system, one or more images or datasets;

provide the one or more images or datasets to the AI Model to obtain an assessment; and

send the assessment to the user, via the user interface.

Similarly in one embodiment an end user computation system for generating an AI based assessment from one or more images or datasets may be provided. The computation system may comprise at least one processor, and at least one memory comprising instructions to configure the at least one processor to:

uploading, via a user interface, an image or dataset to a cloud based Artificial Intelligence (AI) model wherein the AI model is generated according to an embodiment of the methods as described herein; and

receiving the assessment from the cloud based AI model via the user interface.

As discussed above there are many disadvantages of scaling a distributed training regime to support Decentralized Training. Standard distributed training scales poorly with geographic distance and therefore increases the overall cost and turn-around time of training a model by up to two orders of magnitude. This is further exacerbated by the increase in training dataset size as more regions and data sources are connected.

To address this problem, Knowledge Distillation has been described herein to improve the training efficiency of the Decentralized Training framework. Typically, Knowledge Distillation is used to combine ensembles of models to optimize for run time inference performance. However, we propose that the distillation framework can be thought of as being both a Model Parallel and Data Parallel training regime. This instead allows us to train an independent model for each desired locality. Then finally, a student model can be trained for the teachers for each locality.

Embodiments using a simple distillation method, a modified decentralized distillation method, and a specialist and generalist distillation method have been described. Embodiments of these methods enabled training on clusters of centralized datasets, whilst maintaining privacy of each dataset at node. Further embodiments can incorporate the use of compliance checking to ensure that before a trained model is moved away from the node it was trained on, a compliance check is performed to ensure it has generalized appropriately, and has not memorized specific data examples (and thus may constitute a data leak/privacy breach). By using distillation to train the final model from a set of teacher models, we can reduce the overall network cost. For example, the nodes no longer need to synchronize every batch. The modified decentralized distillation method improves over the simple distillation method by allowing a final decentralized training stage may be performed over all the data, for a small number of training iterations. This final step ensures that the model has a chance to be exposed to all the data simultaneously, and mitigate any bias on a specific clinic associated with the ordering of the distillation process, at the cost of additional time and network transfers, but for a much smaller number of epochs compared to training a fully decentralized model. The specialist and generalist distillation method provides further advantages over simple distillation or modified decentralized distillation methods. This approach trains teachers as specialist models, and by exposing them to a sufficient number of locations these specialist models become generalist models. These generalist models can then be combined using ensemble or a further distillation model can be generated from the generalist models. Further, in some embodiments, loss function weighting and under sampling to improve load balancing can be performed, as well as automated provisioning and tearing down of cloud resources. Further the methods may be implemented as multi-level processes to improve scalability.

The method has particular application to datasets where due to data privacy, confidentiality, security, regulatory/legal, or technical reasons, it is not always possible to collect or transfer the data into a single location to create one large and diverse global dataset for the AI to train on. This is particularly true in the health datasets, but it will be understood that the system can be used for other datasets such as security or commercial datasets where similar restrictions apply.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, middleware, platforms, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two, including cloud based systems. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or other electronic units designed to perform the functions described herein, or a combination thereof. Various middleware and computing platforms may be used.

In some embodiments the processor module comprises one or more Central Processing Units (CPUs) or Graphical processing units (GPU) configured to perform some of the steps of the methods. Similarly a computing apparatus may comprise one or more CPUs and/or GPUs. A CPU may comprise an Input/Output Interface, an Arithmetic and Logic Unit (ALU) and a Control Unit and Program Counter element which is in communication with input and output devices through the Input/Output Interface. The Input/Output Interface may comprise a network interface and/or communications module for communicating with an equivalent communications module in another device using a predefined communications protocol (e.g., IEEE 802.11, IEEE 802.15, 4G/5G, TCP/IP, UDP, etc.). The computing apparatus may comprise a single CPU (core) or multiple CPU's (multiple core), or multiple processors. The computing apparatus is typically a cloud based computing apparatus using GPU clusters, but may be a parallel processor, a vector processor, or be a distributed computing device. Memory is operatively coupled to the processor(s) and may comprise RAM and ROM components, and may be provided within or external to the device or processor module. The memory may be used to store an operating system and additional software modules or instructions. The processor(s) may be configured to load and executed the software modules or instructions stored in the memory.

Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by computing device. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

Aspects and features of the various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A method for training an Artificial Intelligence (AI) model on a distributed dataset comprising a plurality of nodes, wherein, and each node comprising a node dataset and the nodes are prevented from accessing other node datasets, comprising:
generating a plurality of trained Teacher models, wherein each Teacher model is a deep neural network model which is locally trained at a node on the node dataset;
moving the plurality of trained Teacher models to a central node, wherein moving a Teacher model comprises transmitting a set of weights representing the Teacher model to the central node;
training a Student model using the plurality of trained Teacher models and a transfer dataset using knowledge distillation.

2. The method as claimed in claim 1, wherein prior to moving the plurality of trained Teacher models to a central node, a compliance check is performed on each trained teacher node to check that the respective model does not contain private data from the node it was trained at by checking if the respective model has memorized specific examples of the data and if the compliance check returns a FALSE value, the respective model is retrained on the data with different parameters until a model that satisfies the compliance check is obtained, or if no model is obtained after N attempts, then either discarding the model or encrypting the model and sharing the model if a data policy allows encrypted sharing of data from the respective node.

3. The method as claimed in claim 1, wherein the transfer dataset is an agreed-upon transfer data drawn from the plurality of node datasets, and/or
the transfer dataset is a distributed dataset comprised of a plurality of node transfer datasets, wherein node transfer dataset is local to a node, or
the transfer dataset is a mixture of agreed-upon transfer data drawn from the plurality of node datasets, and a plurality of node transfer datasets, wherein node local transfer dataset is local to a node.

4. (canceled)

5. (canceled)

6. The method as claimed in claim 1, wherein the nodes exist across separate, geographically isolated localities.

7. The method as claimed in claim 1, wherein the step of training the Student model comprises:
training the Student model using the plurality of trained Teacher models at each of the nodes using the node dataset.

8. The method as claimed in claim 7, wherein prior to training the Student model using the plurality of trained Teacher models, the method further comprises:
forming a single training cluster for training the Student model by establishing a plurality of inter-region peering connections between each of the nodes, and wherein the transfer dataset comprises each of the node datasets, and
wherein after training the Student model at each of the nodes, the Student model is sent to a master node, and copies of the Student model are sent to each of the nodes and assigned as worker nodes, and the master node collects and averages the weights of all worker nodes after each batch to update the Student model.

9. (canceled)

10. The method as claimed in claim 8, wherein prior to sending the Student model to the master node a compliance check is performed on the Student model to check that the Student model does not contain private data from the node it was trained at by checking if the Student model has memorized specific examples of the data and if the compliance check returns a FALSE value, the Student model is retrained on the data with different parameters until a Student model that satisfies the compliance check is obtained, or if no Student model is obtained after N attempts, then either discarding the Student model or encrypting the Student model and sharing the Student model if a data policy allows encrypted sharing of data from the respective node.

11. The method as claimed in claim 1, wherein the step of training the Student model comprises:
training a plurality of Student models, wherein each Student model is a Teacher model at a first node which is trained by a plurality of Teacher models at other nodes by moving the Student model to another node and training the Student model using the Teacher model at the node using the node dataset, and once the plurality of Student models are each trained, an ensemble model is generated from the plurality of trained Student models.

12. The method as claimed in claim 11, wherein prior to training a plurality of Student models, the method further comprises:
forming a single training cluster for training the Student model by establishing a plurality of inter-region peering connections between each of the nodes.

13. The method as claimed in claim 11, wherein prior to moving the Student model to another node a compliance check is performed on the Student model to check that the model does not contain private data from the node it was trained at by checking if the Student model has memorized specific examples of the data and if the compliance check returns a FALSE value, the Student model is retrained on the data with different parameters until a Student model that satisfies the compliance check is obtained, or if no Student model is obtained after N attempts, then either discarding the Student model or encrypting the Student model and sharing the Student model if a data policy allows encrypted sharing of data from the respective node.

14. The method as claimed in claim 11, wherein each Student model is trained after it has been trained at a predetermined threshold number of nodes, or
each Student model is trained after it has been trained on a predetermined quantity of data at at least a threshold number of nodes, or
each Student model is trained after it has been trained at each of the plurality of nodes.

15. (canceled)

16. (canceled)

17. The method as claimed in claim 11, wherein the ensemble model is obtained using an Average Voting method, or
the ensemble model is obtained using weighted averaging, or
the ensemble model is obtained using a Mixture of Experts Layers (learned weighting), or
the ensemble model is obtained using a distillation method, wherein a final model is distilled from the plurality of student models.

18-22. (canceled)

23. The method as claimed in claim 1, further comprising using weighting to adjust a distillation loss function to compensate for differences in the number of data points at each node.

24. (canceled)

25. The method as claimed in claim 1, wherein an epoch comprises a full training pass of each node dataset, and during each epoch, each worker samples a subset of the available sample dataset, wherein the subset size is based a size of the smallest dataset, and the number of epochs is increased based on the ratio of a size of the largest dataset to the size of the smallest dataset.

26. The method as claimed in claim 1, wherein the plurality of nodes are separated into k clusters where k is less than the total number nodes, and the method is performed separately in each cluster to generate k cluster models, wherein each cluster model is stored at a cluster representative node, and the method is performed on the k cluster representative nodes, wherein the plurality of nodes comprises the k cluster representative nodes.

27. The method as claimed in claim 26, wherein one or more additional layers of nodes are created and each lower layer is generated by separating the cluster representative nodes in the previous layer into j clusters where j is less than the number of cluster representative nodes in the previous layer, and then the method is performed separately in each cluster to generate j cluster models, wherein each cluster model is stored at a cluster representative node, and the method is performed on the j cluster representative nodes, wherein the plurality of nodes comprises the j cluster representative nodes.

28. The method as claimed in claim 1, wherein each node dataset is medical dataset comprising one or more medical images or medical diagnostic datasets.

29. The method as claimed in claim 1, further comprising deploying the trained Artificial Intelligence (AI) model.

30. The cloud based computation system as claimed in claim 36, further comprising:
the plurality of local computational nodes, each local computational node comprising one or more processors, one or more memories, one or more network interfaces, and one or more storage devices which store the local node dataset.

31. The system as claimed in claim 30, wherein one or more of the plurality of local computational nodes are cloud based computational nodes.

32. The system as claimed in claim 31, wherein the system is configured to automatically provision the required hardware and software defined networking functionality at at least one of the cloud based computational nodes and the system further comprises:
a cloud provisioning module and a distribution service, wherein the cloud provisioning module is configured to search available server configurations for each of a plurality of cloud services providers, wherein each cloud service provider has a plurality of servers in an associated region, and the cloud provisioning module is configured to apportion a group of servers from one or more of plurality of cloud service providers with tags and metadata to allow a group to be managed, wherein the number of servers in a group is based on number of node locations within a region associated with a cloud service providers, and the distribution service is configured to send a model configuration to a group of servers to begin training a model, and on completion of model training, the provisioning module is configured to shut down the group of servers.

33. (canceled)

34. (canceled)

35. A cloud based computation system for training an Artificial Intelligence (AI) model on a distributed dataset comprising:
at least one cloud based central node comprising one or more processors, one or more memories, one or more network interfaces, and one or more storage devices, wherein the at least one cloud based central node is in communication with a plurality of local computational nodes where each local computational nodes stores a local node dataset wherein access to the local node dataset is limited to the respective computational node, and the least one cloud based central node are configured to train an Artificial Intelligence (AI) model on a distributed dataset formed of the local node datasets by
receiving, by the cloud based central node, a plurality of trained Teacher models from the plurality of local computational nodes wherein each Teacher model is a deep neural network model which is locally trained at a local computational node on the respective local node dataset, and receiving each Teacher model comprises receiving a set of weights representing the Teacher model; and
training a Student model using the plurality of trained Teacher models and a transfer dataset using knowledge distillation.

36. The system as claimed in claim 35 wherein each local node dataset is medical dataset comprising a plurality of medical images and/or medical related test data for performing medical assessments in relation to a patient.

37-40. (canceled)

41. A cloud based computation system for generating an AI based assessment from one or more images or datasets, the cloud based computation system comprising:
one or more computation servers comprising one or more processors and one or more memories configured to store an Artificial Intelligence (AI) model configured to generate an assessment from one or more images or datasets and the one or more computational servers are configured to:
receive, from a user via a user interface of the computational system, one or more images or datasets;
provide the one or more images or datasets to the AI Model to obtain an assessment; and
send the assessment to the user, via the user interface,
wherein the AI model is generated by a cloud based computational training system comprising at least one cloud based central node comprising one or more processors, one or more memories, one or more network interfaces, and one or more storage devices, and a plurality of local computational nodes where each local computational nodes stores a local node dataset wherein access to the local node dataset is limited to the respective local computational node, and the AI model is generated by:
generating a plurality of trained Teacher models, wherein each Teacher model is a deep neural network model which is locally trained at one of the plurality of local computational node on the respective local node dataset;
moving the plurality of trained Teacher models to the at least one cloud based central node, wherein moving a Teacher model comprises transmitting a set of weights representing the Teacher model to the at least one cloud based central node;
training a Student model using the plurality of trained Teacher models and a transfer dataset using knowledge distillation.

42. The system as claimed in claim 41, wherein the one or more image or datasets are medical images and medical datasets and the assessment is a medical assessment of a medical condition, diagnosis or treatment.

43. A computation system for generating an AI based assessment from one or more images or datasets, the computation system comprising at least one processor, and at least one memory comprising instructions to configure the at least one processor to:
upload, via a user interface, an image or dataset to a cloud based Artificial Intelligence (AI) model configured to generate an assessment from one or more images or datasets; and
receive the assessment from the cloud based AI model via the user interface,
wherein the AI model is generated by:
generating a plurality of trained Teacher models, wherein each Teacher model is a deep neural network model which is locally trained at a local computational node on the local node dataset;
moving the plurality of trained Teacher models to the central computational node, wherein moving a Teacher model comprises transmitting a set of weights representing the Teacher model to the central node; and
training a Student model using the plurality of trained Teacher models and a transfer dataset using knowledge distillation.

44. The system as claimed in claim 43, wherein the one or more image or datasets are medical images and medical datasets and the assessment is a medical assessment of a medical condition, diagnosis or treatment.
